(19) United States
(12) Patent Application Publication
(10) Pub. No.: US 2025/0295488 A1
(43) Pub. Date: Sep. 25, 2025

(54) CAVAL DOCKING IMPLANTS

(71) Applicant: EDWARDS LIFESCIENCES CORPORATION, Irvine, CA (US)

(72) Inventors: Michael G. Valdez, Riverside, CA (US); Omar Fawzi Azanki, Irvine, CA (US); Anne Bernadette Aragon Alcasid, Costa Mesa, CA (US); David Lyle Leibowitz, Capistrano Beach, CA (US); Tiana (formerly Thu Thi Anh) Tran, Westminster, CA (US); John Charles Laschinger, Laguna Beach, CA (US); Michael R. White, Orange, CA (US); Afshin Moien Jahromi, Orange, CA (US); Amirsepehr Azimian, Costa Mesa, CA (US)

(73) Assignee: EDWARDS LIFESCIENCES CORPORATION, Irvine, CA (US)

(21) Appl. No.: 19/229,809

(22) Filed: Jun. 5, 2025

Related U.S. Application Data
(63) Continuation of application No. PCT/US2023/084350, filed on Dec. 15, 2023.
(60) Provisional application No. 63/476,103, filed on Dec. 19, 2022.

(51) Int. Cl. A61F 2/24

(52) U.S. Cl. CPC A61F 2/2418; A61F 2220/0075; A61F 2230/0067; A61F 2230/0069; A61F 2230/0071; A61F 2250/0039; A61F 2250/006; A61F 2250/0069

(57) ABSTRACT

A cardiac implant device may include a frame sized for placement at least partially within a blood vessel of a heart, the frame having a proximal end, a distal end, and a midsection, and comprising an outer frame and an inner frame. At least a portion of the inner frame is joined to the outer frame at the distal end of the frame and wherein the inner frame extends away from the outer frame at the midsection of the frame to form a gap between the inner frame and the outer frame. A device may include a sealing skirt extending at least partially along the inner frame and the distal end of the frame.

### RELATED APPLICATION

This application is a continuation of International Patent Application No. PCT/US2023/084350, filed Dec. 15, 2023, which claims the benefit of U.S. Provisional Application No. 63/476,103, filed Dec. 19, 2022, the complete disclosures of which are hereby incorporated by reference in their entireties.

### BACKGROUND

### Field

The present disclosure generally relates to the field of a medical implant devices.

### Description of Related Art

Various medical procedures involve the implantation of medical implant devices within the anatomy of the heart. Certain physiological parameters associated with such anatomy, such as fluid pressure, can have an impact on patient health prospects.

### SUMMARY

Described herein are one or more methods and/or devices to facilitate monitoring of physiological parameter(s) associated with certain chambers and/or vessels of the heart, such as the left atrium, using one or more sensor implant devices.

For purposes of summarizing the disclosure, certain aspects, advantages, and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart in accordance with one or more examples.

FIGS. 2A-2D illustrate components of an example implant configured to dock and/or support one or more prosthetic valves and/or valve components in accordance with one or more instances.

FIGS. 3A-3D illustrate components of an example implant configured to dock and/or support one or more prosthetic valves and/or valve components in accordance with one or more instances.

FIG. 4 illustrates an example implant configured to dock and/or support one or more prosthetic valves and/or valve components in accordance with one or more instances.

FIG. 5 illustrates components of an example implant configured to dock and/or support one or more prosthetic valves and/or valve components in accordance with one or more instances.

FIGS. 6A and 6B illustrate components of an example implant configured to dock and/or support one or more prosthetic valves and/or valve components in accordance with one or more instances.

FIGS. 7A-7D illustrate components of an example implant configured to dock and/or support one or more prosthetic valves and/or valve components in accordance with one or more instances.

FIG. 8 illustrates components of an example implant 800 implanted within a blood vessel (e.g., the IVC) and/or configured to dock one or more prosthetic valves 805 and/or valve components in accordance with one or more instances.

FIG. 9 illustrate components of an example implant implanted within a blood vessel (e.g., the IVC) and/or configured to dock one or more prosthetic valves and/or valve components in accordance with one or more instances.

FIGS. 10A and 10B illustrate components of an example implant configured to dock and/or support one or more prosthetic valves and/or valve components in accordance with one or more instances.

FIG. 11 illustrates another example implant configured for anchoring into one or more blood vessels and/or docking one or more prosthetic valves in accordance with one or more examples.

FIG. 12 illustrates an example implant anchored into one or more blood vessels (e.g., the IVC) and/or configured to dock one or more prosthetic valves in accordance with one or more examples.

FIG. 13 illustrates another example implant configured for placement at least partially within a blood vessel (e.g., the IVC) and/or configured to dock one or more prosthetic valves in accordance with one or more examples herein.

FIG. 14 illustrates another example implant disposed within a blood vessel in accordance with one or more examples.

FIG. 15 illustrates another example implant disposed within a blood vessel in accordance with one or more examples.

FIG. 16 illustrates another example implant disposed within a blood vessel in accordance with one or more examples.

FIG. 17 illustrates components of another example implant configured to dock and/or support one or more prosthetic valves and/or valve components in accordance with one or more instances.

FIG. 18 illustrates components of another example implant configured to dock and/or support one or more prosthetic valves and/or valve components in accordance with one or more instances.

FIGS. 19A and 19B illustrate an outer frame of an example implant configured to dock and/or support one or more prosthetic valves and/or valve components in accordance with one or more instances.

FIGS. 20A and 20B illustrate an inner frame of an example implant configured to dock and/or support one or more prosthetic valves and/or valve components in accordance with one or more instances.

FIG. 21 illustrates at least a portion of an example arm of an implant (e.g., an outer frame and/or inner frame of the implant), in accordance with one or more examples herein.

FIG. 22 provides a cross-sectional side view of an example implant configured to dock and/or support one or more prosthetic valves and/or valve components in accordance with one or more instances.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred examples. Although certain spatially relative terms, such as “outer,” “inner,” “cupper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for delivery of prosthetic heart valves and/or for managing blood flow within a heart. In some implementations, the present disclosure relates to cardiac implant devices that incorporate or are associated with prosthetic valves. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain examples are disclosed herein in the context of cardiac implant devices. However, although certain principles disclosed herein are particularly applicable to the anatomy of the heart, it should be understood that sensor implant devices in accordance with the present disclosure may be implanted in, or configured for implantation in, any suitable or desirable anatomy.

In some examples, an implant device may comprise one or more markers, including radiopaque markers, to facilitate visualization and/or location of the sensor device within a patient's body. A marker may be embedded within a frame and/or sealing element of the implant of the device.

Some implementations of the present disclosure relate to stents and/or similar devices configured for managing blood flow through one or more blood vessels. Some examples relate to stents and/or similar devices comprising prosthetic valves and/or prosthetic valve components (e.g., leaflets) and/or configured for docking a prosthetic valve.

Some examples comprise distal flared ends and/or compressible flanges disposed at or near distal ends of frames of example stent devices. In some examples, a portion of an implant (e.g., a flange and/or distal end) may be configured to extend out of a blood vessel and/or at least partially along a floor of a chamber (e.g., right atrium) of the heart. Some components of example implants may be at least partially enclosed and/or covered by a covering comprising one or more cloths, fabrics, and/or other materials. In some examples, a covering may be configured to increase frictional contact and/or increase friction between an implant and the native anatomy.

Some example implants may comprise various features and/or components configured to minimize contact and/or blockage of one or more branching blood vessels (e.g., hepatic veins). For example, one or more implants may comprise inwardly bent walls and/or inward bulges at or near a midsection of the implant such that the implant recedes from the walls of a main blood vessel and/or from an inflow junction of one or more branching blood vessels. In some examples, an implant may comprise a network of wires and/or struts configured to form cells having any suitable shapes and/or sizes. In some examples, one or more cells at or near a midsection of the implant may be larger than cells at other portions of the implant.

While some implants described herein may be configured to receive and/or dock one or more prosthetic valves, some example implants may comprise incorporated valves and/or valve components (e.g., leaflets). For example, any of the implants described herein may comprise coupled and/or affixed leaflets configured to extend across lumens of the implants (e.g., at distal ends of the implants). In some examples, leaflets may extend from a frame of the implant. The frame may have a compressible, expandible, and/or flexible structure.

Example implants can comprise various features for anchoring to the native anatomy. In some examples, an implant may comprise one or more inward and/or outward extending arms. Some example anchoring arms may extend longitudinally and/or laterally from a frame of the implant. In some examples, one or more anchoring arms may comprise various anchoring features, which can include barbed ends and/or similar features.

The present disclosure relates to implants in one or more blood vessels, including inferior vena cava and/or superior vena cava. Some example implants may comprise laser-cut nitinol tube frames and/or a sealing skirt attached at one end (e.g., at a distal end configured to extend into the right atrium and/or other chamber). Some implants may be relatively long and/or may comprise a sealing skirt that extends a sufficient distance such that, for some patients, the sealing skirt can at least partially occlude one or more branching blood vessels (e.g., the hepatic veins). Because of the variation in exactly where the hepatic veins and/or other branching blood vessels enter the vena cava and/or other blood vessels, some implants described herein may be applicable for variably sized and/or shaped anatomies.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

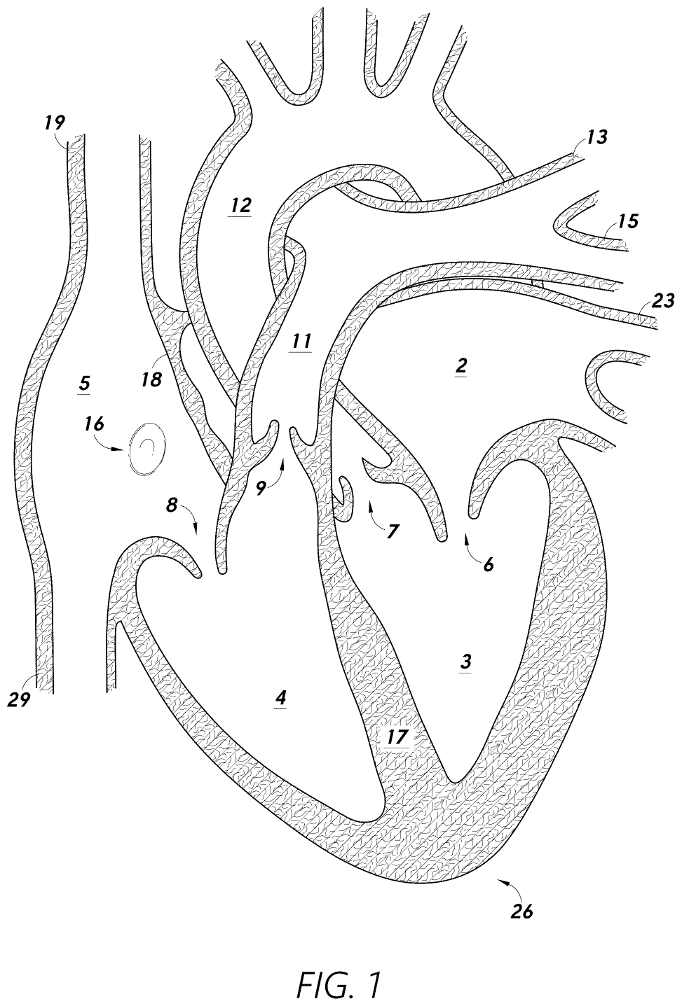

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. The pulmonary veins 23 carry blood from the lungs to the left atrium 2.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Dysfunction of a heart valve and/or associated leaflets (e.g., pulmonary valve dysfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle, referred to as the septum, separates the left-side chambers from the right-side chambers. In particular, an atrial septum wall portion 18 (referred to herein as the “atrial septum,” “interatrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 26 of the heart 1 is referred to as the apex and is generally located on or near the midclavicular line, in the fifth intercostal space.

The coronary sinus 16 comprises a collection of veins joined together to form a large vessel that collects blood from the heart muscle (myocardium). The ostium of the coronary sinus 16, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

Any of several access pathways in the heart 1 may be utilized for maneuvering guidewires and catheters in and around the heart 1 to deploy implants and/or devices of the present application. For instance, access may be from above via either the subclavian vein or jugular vein into the superior vena cava (SVC) 19, right atrium 5, and from there into the coronary sinus 16. Alternatively, the access path may start in the femoral vein and through the inferior vena cava (IVC) 14 into the heart 1. Other access routes may also be used, and each can utilize a percutaneous incision through which the guidewire and catheter are inserted into the vasculature, normally through a sealed introducer, and from there the physician can control the distal ends of the devices from outside the body.

### Caval Implant Devices

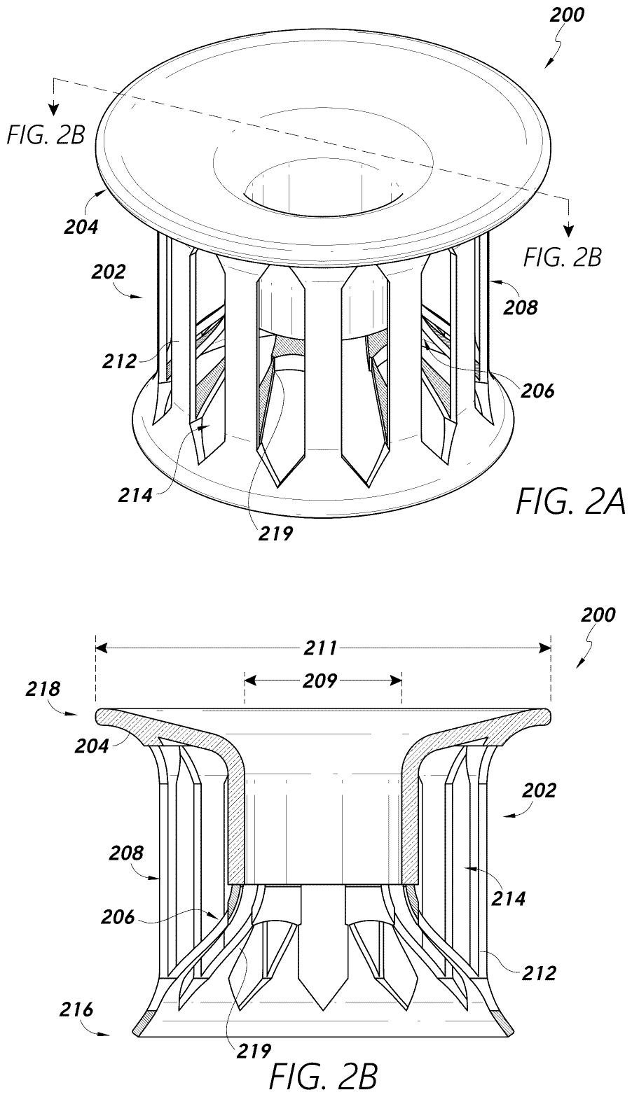

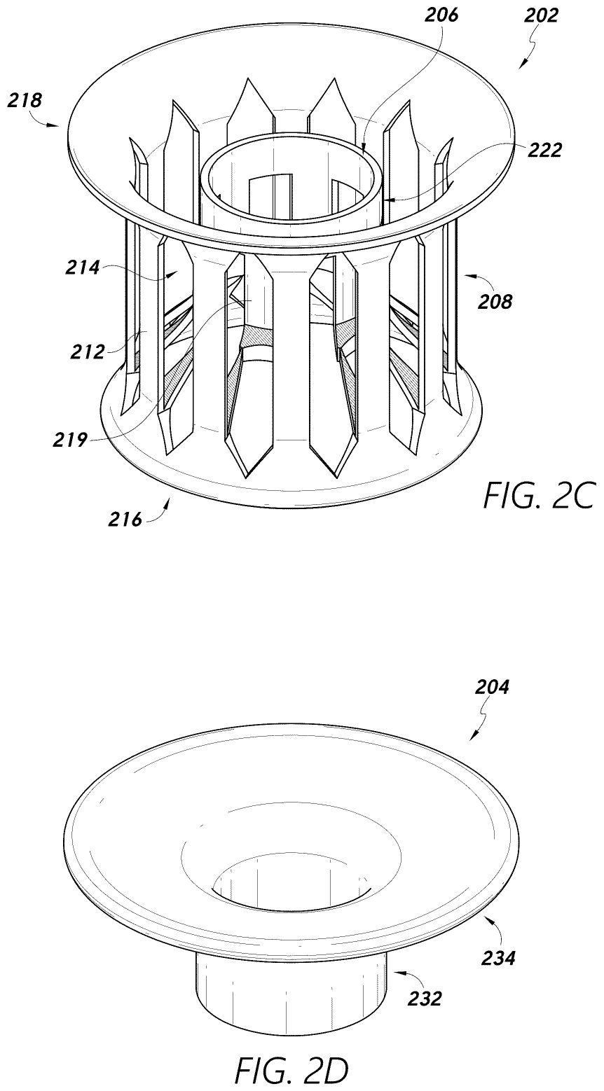

FIGS. 2A-2D illustrate components of an example implant 200 configured to dock and/or support one or more prosthetic valves and/or valve components in accordance with one or more instances. The implant 200 may comprise a frame 202 and/or a sealing element 204, which can include a skirt, covering, and/or similar device. FIG. 2A provides a perspective view of the implant 200. FIG. 2B provides a cross-sectional side view of the implant 200. FIG. 2C provides a perspective view of the frame 202 of the implant 200. FIG. 2D provides a perspective view of the sealing element 204 of the implant 200. In some examples, the implant 200 may be configured for placement at a defective heart valve and/or an ostium of a blood vessel (e.g., at a Thebesian valve).

The frame 202 may be configured to form an inner frame 206 and/or an outer frame 208. The inner frame 206 may form a first diameter 209 that is less than a second diameter 211 of the outer frame 208. The inner frame 206 and outer frame 208 may be extensions of a common device and/or may extend from each other.

In some examples, the outer frame 208 may have a generally cylindrical form and/or may at least partially enclose a complete circumference of the inner frame 206 and/or of a portion of the sealing element 204. The outer frame 208 may comprise a network of struts 212, which can include wires, arms, bars, cords, walls, and/or similar components forming one or more cells 214 and/or openings through the outer frame 208. The one or more cells 214 may be configured to allow blood flow through the outer frame 208. The cells 214 may have any suitable shape and/or size. In the example shown in FIGS. 2A and 2B, the outer frame 208 may form generally elongate cells 214 extending approximately an entire length of the outer frame 208 and/or from a proximal end 216 to a distal end 218 of the implant 200. The one or more cells 214 may have triangular forms at end points of the one or more cells 214, as shown in FIGS. 2A and 2B. However, the one or more struts 212 may form cells 214 having different shapes. For example, the struts 212 may be configured to form generally rectangular and/or diamond-shaped cells 214. While the struts 212 are shown having generally thick structures, the one or more struts 212 may have wire-like and/or generally thin forms. In some examples, the inner frame 206 and/or outer frame 208 may be configured to maintain a uniform structure and/or strut 212 pattern along a length of the frame 202.

The implant 200 may be configured for delivery and/or placement at an SVC and/or IVC of a heart. For example, the implant 200 may be configured for placement at or near an in-flow junction of the SVC and/or IVC to the right atrium of the heart.

In some examples, the implant 200 may have a relatively shorter (e.g., between the proximal end 216 and the distal end 218 of the implant 200) form relative to other caval implants. The frame 202 may be at least partially composed of one or more shape memory alloys (e.g., Nitinol) and/or may be laser-cut into a desired form. In some examples, the frame 202 and/or sealing element 204 may have a generally flared, mushroom, and/or funnel shape and/or form at or near the distal end 218 of the implant 200. For example, the distal end 218 of the implant 200 may be flared and/or conical in shape to extend outward into the right atrium and/or to form a grip onto a rim of the right atrium. In some examples, the sealing element 204 may be configured to extend beyond the frame 202 and/or may form an extension of the frame 202 to extend further laterally and/or longitudinally relative to the frame 202. In some examples, the outer frame 208 may be at least partially flared at or near the distal end 218 of the implant 200. The inner frame 206 may have a generally straight form extending towards the distal end 218 and/or may flare outwardly towards the outer frame 208 at or near the proximal end 216.

The proximal end 216 may have a straight or flared form. In the example shown in FIGS. 2A and 2B, the inner frame 206 and outer frame 208 may join together at or near the proximal end 216 and/or may flare outwardly to improve a proximal seal of the implant 200. Alternatively, the frame 202 may be configured to flare inwardly at or near the proximal end 216 and/or may be configured to taper inwardly to approximate a shaped of a blood vessel. For example, the outer frame 208 may have a generally tapered and/or inwardly angled form extending from the distal end 218 to the proximal end 216.

In some examples, the inner frame 206 may comprise a network of struts 212 forming one or more cells 214, similarly to the outer frame 208. The inner frame 206 may comprise one or more elongate arms 219, prongs, fingers, and/or similar device extending from the frame 202 at or near the proximal end 216 and/or a midsection of the frame 202. The one or more arms 219 may be configured to join together to form a generally circular, tubular, and/or cylindrical midsection 222 of the frame 202. A prosthetic valve and/or the sealing element 204 may be configured to be mounted to the midsection 222. In some examples, the one or more arms 219 may not be interconnected and/or may comprise free ends configured to support the sealing element 204. Alternatively, the one or more arms 219 may be configured to flare outwardly at or near the distal end 218 of the implant 200 and/or to join the outer frame 208 at or near the distal end 218.

The inner frame 206 may be configured to extend outwardly and/or towards the proximal end 216. The outer frame 208 and inner frame 206 may form a V-shaped juncture at or near the proximal end 216. The implant 200 may form a flared end at the distal end 218 and/or proximal end 216 to engage an atrium and/or other anatomy when implanted. The sealing element 204 may be configured to extend from the flared distal end 218 and/or may extend at least partially along an inner and/or outer surface of the inner frame 206. In some examples, the sealing element 204 may be configured to bridge a gap and/or space between the inner frame 206 and the outer frame 208 at or near the distal end 218 of the implant 200.

The implant 200 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 204 may not extend along a complete length of the frame 202. For example, the sealing element 204 may extend from the distal end 218 of the implant 200 to a midsection and/or midpoint of the implant 200 and/or inner frame 206. In some examples, the sealing element 204 may not extend to a junction between the inner frame 206 and outer frame 208 at or near the proximal end 216 of the implant 200. Moreover, the sealing element 204 may not extend over an outwardly extending portion of the inner frame 206 and/or may only extend along a generally straight segment of the inner frame 206. The implant 200 may be configured such that, when implanted at the junction between the IVC and the right atrium, the sealing element 204 may not extend over the one or more hepatic veins and/or branching blood vessels. In some examples, the frame 202 may at least partially extend over the hepatic veins and/or branching blood vessels and/or may allow blood flow from the branching blood vessels through cells 214 of the frame 202. The inner frame 206 may be configured to hold the sealing element 204 distally from the outer frame 208 and/or walls of the blood vessel such that the sealing element 204 may not create blockage of one or more branching blood vessels if the sealing element 204 does extend across one or more branching blood vessels.

In some examples, the implant 200 may comprise one or more radiopaque markers configured to facilitate delivery and/or use of the implant 200. For example, one or more markers may be disposed along the inner frame 206 (e.g., at a midsection of the inner frame 206 and/or at a point at which the sealing element 204 ends) to assist with aligning and/or docking one or more prosthetic valves at the implant 200 and/or inner frame 206. In another example, the implant 200 may comprise one or more markers at the outer frame 208 and/or sealing element 204 at or near the distal end 218 to facilitate positioning of the implant 200 and/or distal end 218 at the atrium junction and/or other anatomy.

In some examples, the frame 202 and/or outer frame 208 may comprise one or more elongate arms and/or fingers configured to flare out at the distal end 218. The one or more arms and/or fingers may be configured to support a flared end of the sealing element 204 and/or to approximate a curvature of the junction between the blood vessel and right atrium and/or other chamber. The sealing element 204 may be composed of any suitable material, which can include fabric (e.g., polyester), cloth, and/or rubber. The one or more arms and/or fingers may be configured extend under the sealing element 204 to hold the sealing element 204 up and/or otherwise support the sealing element 204. In some examples, the sealing element 204 may be configured to extend over and/or under the frame 202 and/or arms at the distal end 218. For example, the sealing element 204 may be configured to wrap around the frame 202 and/or extend between the frame 202 at the distal end 218 and the native anatomy to improve sealing of the frame 202 with the anatomy.

The frame 202 may be coupled to the sealing element 204 and/or may be configured to mate with the sealing element 204. In some examples, one or more sutures and/or other attachment features (e.g., clips, hooks, notches, stitches, adhesives, etc.) may be used to form an attachment between the frame 202 and the sealing element 204.

In some examples, the implant 200 may be configured to cause native anatomy to conform to a size and/or shape of the implant 200. For example, the outer frame 208 and/or distal end 218 of the implant 200 may be configured to press outwardly against the native tissue to create a larger opening to support a prosthetic valve. In some examples, the implant 200 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 202. In some examples, the frame 202 may increase in diameter at the bulb and/or may decrease in diameter on either side of the bulb.

The outer frame 208 and/or inner frame 206 may be configured to form relatively large cells 214. For example, a majority of a surface area of the outer frame 208 and/or inner frame 206 may be empty space between struts 212 of the outer frame 208 and/or inner frame 206.

The sealing element 204 (e.g., skirt) may have any suitable size and/or shape. In some examples, the sealing element 204 can comprise a proximal portion 232 having a smaller width and/or diameter than a distal portion 234 of the sealing element 204. The proximal portion 232 may have a tubular and/or cylindrical form. The distal portion 234 may flare out from the proximal portion 232 and/or may gradually extend into a larger diameter to provide a distal sealing end of the implant 200. The sealing element 204 may form a funnel and/or mushroom shape around an inner lumen extending through the sealing element 204 (e.g., at a central portion of the sealing element 204). In some examples, the sealing element 204 may comprise a disc shape (e.g., at least at the distal portion 234).

The arms 219 of the implant 200 may extend upwardly from the proximal end 216 to the distal end 218. For example, the one or more arms 219 may extend at an approximately 45-degree angle from the outer frame 208 at or near the distal end 218 of the implant 200 and/or may generally upwardly generally in parallel with the outer frame 208 along a midsection of the implant 200.

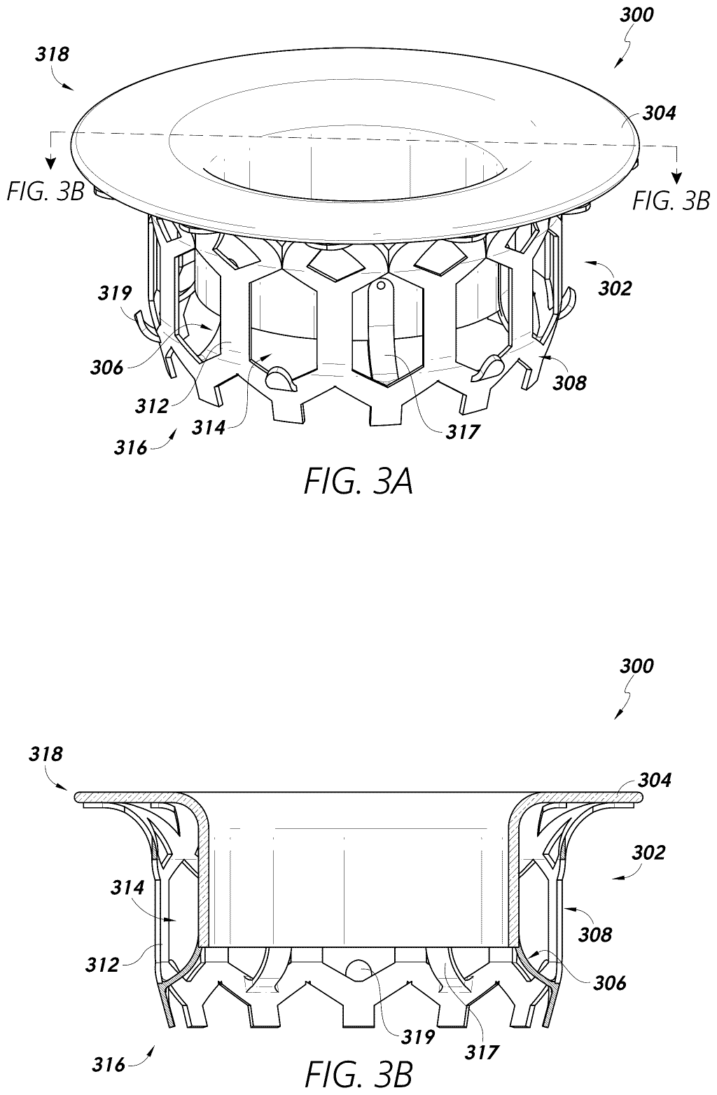

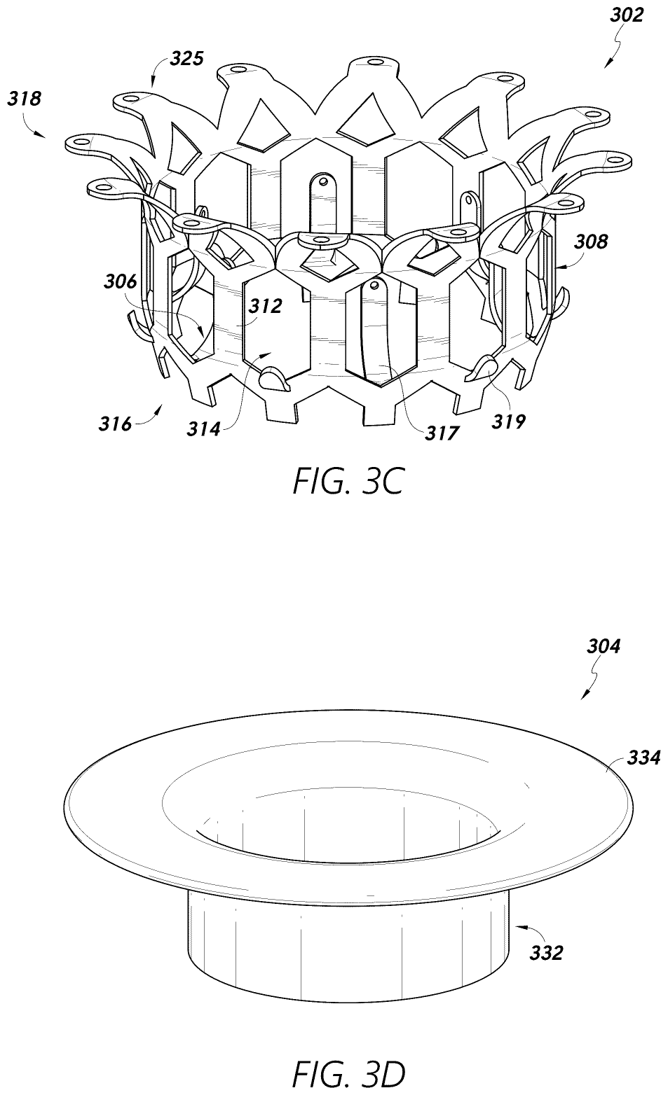

FIGS. 3A-3D illustrate components of an example implant 300 configured to dock and/or support one or more prosthetic valves and/or valve components in accordance with one or more instances. The implant 300 may comprise a frame 302 and/or a sealing element 304, which can include a skirt, covering, and/or similar device. The frame 302 may be configured to form an inner frame 306 and/or an outer frame 308. The inner frame 306 and outer frame 308 may be extensions of a common device and/or may extend from each other. FIG. 3A provides a perspective view of the implant 300. FIG. 3B provides a cross-sectional side view of the implant 300. FIG. 3C provides a perspective view of the frame 302 of the implant 300. FIG. 3D provides a perspective view of the sealing element 304 of the implant 300.

In some examples, the outer frame 308 may have a generally cylindrical form and/or may at least partially enclose a complete circumference of the inner frame 306 and/or of a portion of the sealing element 304. The outer frame 308 may comprise a network of struts 312, which can include wires, arms, bars, cords, walls, and/or similar components forming one or more cells 314 and/or openings through the outer frame 308. The one or more cells 314 may be configured to allow blood flow through the outer frame 308. The cells 314 may have any suitable shape and/or size. In the example shown in FIGS. 3A and 3B, the outer frame 308 may form generally elongate cells 314 extending approximately an entire length of the outer frame 308 and/or from a proximal end 316 to a distal end 318 of the implant 300. The one or more cells 314 may have triangular forms at end points of the one or more cells 314, as shown in FIGS. 3A and 3B. However, the one or more struts 312 may form cells 314 having different shapes. For example, the struts 312 may be configured to form generally rectangular and/or diamond-shaped cells 314. While the struts 312 are shown having generally thick structures, the one or more struts 312 may have wire-like and/or generally thin forms. In some examples, the inner frame 306 and/or outer frame 308 may be configured to maintain a uniform structure and/or strut 312 pattern along a length of the frame 302.

The implant 300 may be configured for delivery and/or placement at an SVC and/or IVC of a heart. For example, the implant 300 may be configured for placement at or near an in-flow junction of the SVC and/or IVC to the right atrium of the heart.

The inner frame 306 may comprise one or more elongate fingers 317 (e.g., prongs, arms, etc.) extending inwardly and/or into an inner lumen formed by the outer frame 308. In some examples, the outer frame 308 may comprise struts 312 forming generally stretched and/or hexagonal cells 314. Each of the fingers 317 may extend from below a cell 314 of the outer frame 308. The frame 302 may additionally or alternatively comprise one or more anchors 319 (e.g., prongs, finger, arms, etc.) extending outwardly and/or away from the inner lumen formed by the outer frame 308. In some examples, the one or more anchors 319 may extend from below cells 314 of the outer frame 308. The fingers 317 and anchors 319 may extend from alternating cells 314 of the outer frame 308. For example, each finger 317 may be disposed between two anchors 319 and/or each anchor 319 may be disposed between two fingers 317. The fingers 317 and/or anchors 319 may have generally curved forms and/or may extend upwardly towards a distal end 318 of the implant 300 while also extending outwardly and/or inwardly relative to the inner lumen formed by the outer frame 308. In some examples, the fingers 317 may be longer and/or may extend further towards the distal end 318 than the anchors 319.

The one or more fingers 317 may be configured to support the sealing element 304. For example, the sealing element 304 may be configured to attach to and/or extend along an inner and/or outer surface of the one or more fingers 317. The one or more fingers 317 may be configured to hold the sealing element 304 at a distance from the outer frame 308.

The one or more anchors 319 may be configured to extend outwardly into contact with tissue and/or to penetrate surrounding tissue to anchor the implant 300 within a blood vessel. While the anchors 319 are shown extending upwardly (e.g., towards a distal end 318 of the implant 300), one or more anchors 319 may additionally or alternatively extend downwards (e.g., towards a proximal end 316 of the implant 300).

The frame 302 may comprise one or more flaring arms 325 at or near the distal end 318 of the implant 300. For example, the one or more flaring arms 325 may comprise curved and/or bent struts 312 of the outer frame 308 extending generally outwardly to facilitate anchoring of the implant 300 along a floor of a chamber of a heart. In some examples, one or more arms 325 may comprise apertures and/or holes configured to receive one or more sutures and/or other delivery systems.

The sealing element 304 (e.g., skirt) may have any suitable size and/or shape. In some examples, the sealing element 304 can comprise a proximal portion 332 having a smaller width and/or diameter than a distal portion 334 of the sealing element 304. The proximal portion 332 may have a tubular and/or cylindrical form. The distal portion 334 may flare out from the proximal portion 332 and/or may gradually extend into a larger diameter to provide a distal sealing end of the implant 300.

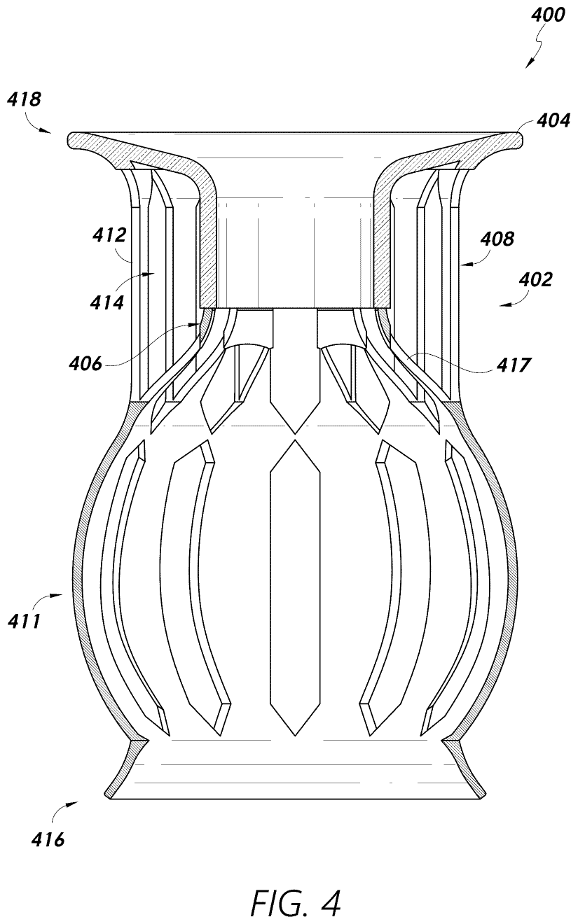

FIG. 4 illustrates an example implant 400 configured to dock and/or support one or more prosthetic valves and/or valve components in accordance with one or more instances. The implant 400 may comprise a frame 402 and/or a sealing element 404, which can include a skirt, covering, and/or similar device. The frame 402 may be configured to form an inner frame 406 and/or an outer frame 408. The inner frame 406 and outer frame 408 may be extensions of a common device and/or may extend from each other.

In some examples, the outer frame 408 may have a generally cylindrical form and/or may at least partially enclose a complete circumference of the inner frame 406 and/or of a portion of the sealing element 404. The outer frame 408 may comprise a network of struts 412, which can include wires, arms, bars, cords, walls, and/or similar components forming one or more cells 414 and/or openings through the outer frame 408. The one or more cells 414 may be configured to allow blood flow through the outer frame 408. The cells 414 may have any suitable shape and/or size. In the example shown in FIGS. 4A and 4B, the outer frame 408 may form generally elongate cells 414 extending approximately an entire length of the outer frame 408 and/or from a proximal end 416 to a distal end 418 of the implant 400. The one or more cells 414 may have triangular forms at end points of the one or more cells 414, as shown in FIGS. 4A and 4B. However, the one or more struts 412 may form cells 414 having different shapes. For example, the struts 412 may be configured to form generally rectangular and/or diamond-shaped cells 414. While the struts 412 are shown having generally thick structures, the one or more struts 412 may have wire-like and/or generally thin forms. In some examples, the inner frame 406 and/or outer frame 408 may be configured to maintain a uniform structure and/or strut 412 pattern along a length of the frame 402.

The implant 400 may be configured for delivery and/or placement at an SVC and/or IVC of a heart. For example, the implant 400 may be configured for placement at or near an in-flow junction of the SVC and/or IVC to the right atrium of the heart.

In some examples, the frame 402 can have a relatively large length and/or may be configured to extend relatively far into the IVC and/or other blood vessel. The frame 402 can comprise an outward bulb 411 configured to increase a diameter of the frame 402 to facilitate anchoring the implant 400 within the IVC and/or other blood vessel. The implant 400 may have an hourglass shape and/or may be configured to increase in diameter at the outward bulb 411 and/or to decrease in diameter on either side of the outward bulb 411.

The inner frame 406 and outer frame 408 may be configured to join at a midsection of the frame 402. For example, the inner frame 406 and the outer frame 408 may join above the outward bulb 411. The sealing element 404 may be configured to extend along the inner frame 406 and/or may not extend into the outward bulb 411 portion of the frame 402.

The frame 402 may comprise one or more upward-extending arms 417 configured to form the inner frame 406. For example, the arms 417 may extend upwardly from the outer frame 408 at or near a midsection of the frame 402 and/or above the bulb 411 of the frame 402. The one or more arms 417 may be configured to extend at an acute angle away from the outer frame into the lumen of the frame 402 and/or may extend generally in parallel with the outer frame 408 at or near distal ends of the one or more arms 417.

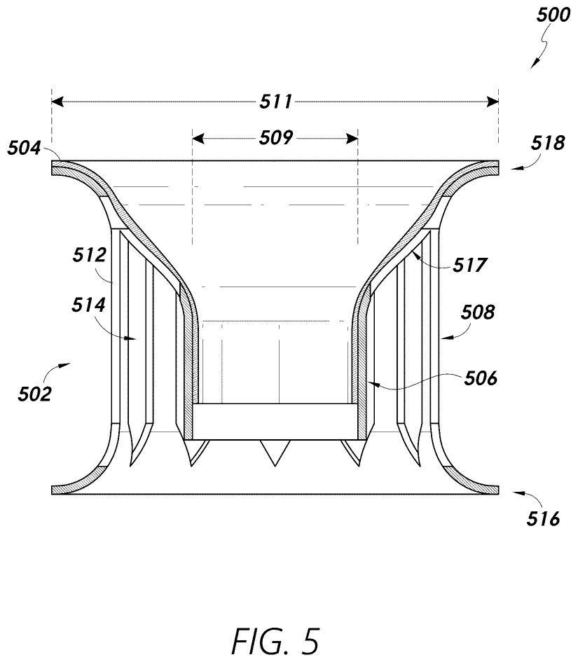

FIG. 5 illustrates components of an example implant 500 configured to dock and/or support one or more prosthetic valves and/or valve components in accordance with one or more instances. The implant 500 may comprise a frame 502 and/or a sealing element 504, which can include a skirt, covering, and/or similar device. The frame 502 may be configured to form an inner frame 506 and/or an outer frame 508. The inner frame 506 may form a first diameter 509 that is less than a second diameter 511 of the outer frame 508. The inner frame 506 and outer frame 508 may be extensions of a common device and/or may extend from each other.

In some examples, the outer frame 508 may have a generally cylindrical form and/or may at least partially enclose a complete circumference of the inner frame 506 and/or of a portion of the sealing element 504. The outer frame 508 may comprise a network of struts 512, which can include wires, arms, bars, cords, walls, and/or similar components forming one or more cells 514 and/or openings through the outer frame 508. The one or more cells 514 may be configured to allow blood flow through the outer frame 508. The cells 514 may have any suitable shape and/or size. The outer frame 508 may form generally elongate cells 514 extending approximately an entire length of the outer frame 508 and/or from a proximal end 516 to a distal end 518 of the implant 500. The one or more cells 514 may have triangular forms at end points of the one or more cells 514. However, the one or more struts 512 may form cells 514 having different shapes. For example, the struts 512 may be configured to form generally rectangular and/or diamond-shaped cells 514. While the struts 512 are shown having generally thick structures, the one or more struts 512 may have wire-like and/or generally thin forms. In some examples, the inner frame 506 and/or outer frame 508 may be configured to maintain a uniform structure and/or strut 512 pattern along a length of the frame 502.

The implant 500 may be configured for delivery and/or placement at an SVC and/or IVC of a heart. For example, the implant 500 may be configured for placement at or near an in-flow junction of the SVC and/or IVC to the right atrium of the heart.

At a distal end 518 of the implant 500, the outer frame 508 and inner frame 506 may join together and/or the inner frame 506 may extend away from the outer frame 508 and/or along an inner lumen formed by the outer frame 508. In some examples, the outer frame 508 and inner frame 506 may both have a generally flared and/or conical form at or near the distal end 518 of the implant 500. The inner frame 506 may have a flaring angle that is greater than the outer frame 508 such that the diameter of the inner frame 506 may be less than the diameter of the outer frame 508. The inner frame 506 may be configured to extend along at least a portion of the length of the outer frame 508.

The flared end (e.g., distal end 518) of the outer frame 508 and/or inner frame 506 may be configured to engage an atrium and/or other chamber when implanted. The sealing element 504 may be configured to extend along the inner frame 506 and/or may be configured to wrap around the outer frame 508 and/or extend between the outer frame 508 and the native tissue. The outer frame 508 may be configured to extend into the IVC and/or other blood vessel and/or the sealing element 504 may be configured to extend from the outer frame 508 to an inner surface of the inner frame 506. The sealing element 504 may be configured for engagement with a prosthetic valve and/or other implant. For example, the sealing element 504 may be configured to provide a mounting surface for a prosthetic valve and/or may be configured to securely hold the prosthetic valve. The sealing element 504 may be configured to extend along only a portion of the implant 500 such that the sealing element 504 may not extend across one or more branching vessels of the blood vessel.

In some examples, the implant 500 may comprise one or more outward bulbs configured to extend outwardly from the diameter of the outer frame 508 to facilitate anchoring of the implant 500 within a blood vessel.

The frame 502 may comprise one or more downward-extending arms 517 (e.g., extending towards the proximal end 516) configured to form the inner frame 506. For example, the arms 517 may extend downwardly from the outer frame 508 at or near the distal end 518 of the frame 502. The one or more arms 517 may be configured to extend at an acute angle away from the outer frame into the lumen of the frame 502 and/or may extend generally in parallel with the outer frame 508 at or near distal ends of the one or more arms 517.

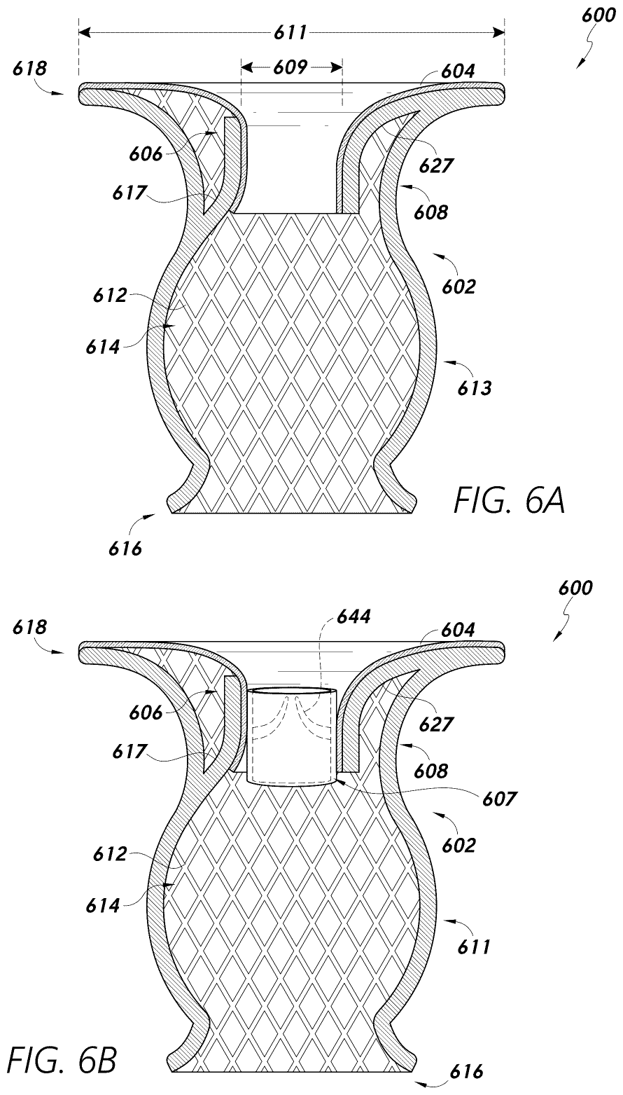

FIGS. 6A and 6B illustrate components of an example implant 600 configured to dock and/or support one or more prosthetic valves and/or valve components in accordance with one or more instances. FIG. 6A provides a cross-sectional side view of the implant 600. FIG. 6B provides a cross-sectional side view of the implant 600 with a prosthetic valve 607 docked at the inner frame 606 of the implant 600 and/or within a lumen of the frame 602 of the implant 600. The implant 600 may comprise a frame 602 and/or a sealing element 604, which can include a skirt, covering, and/or similar device. The frame 602 may be configured to form an inner frame 606 and/or an outer frame 608. The inner frame 606 may form a first diameter 609 that is less than a second diameter 611 of the outer frame 608. The inner frame 606 and outer frame 608 may be extensions of a common device and/or may extend from each other.

In some examples, the outer frame 608 may have a generally cylindrical form and/or may at least partially enclose a complete circumference of the inner frame 606 and/or of a portion of the sealing element 604. The outer frame 608 may comprise a network of struts 612, which can include wires, arms, bars, cords, walls, and/or similar components forming one or more cells 614 and/or openings through the outer frame 608. The one or more cells 614 may be configured to allow blood flow through the outer frame 608. The cells 614 may have any suitable shape and/or size. In the example shown in FIGS. 6A and 6B, the outer frame 608 may form generally elongate cells 614 extending approximately an entire length of the outer frame 608 and/or from a proximal end 616 to a distal end 618 of the implant 600. The one or more cells 614 may have triangular forms at end points of the one or more cells 614, as shown in FIGS. 6A and 6B. However, the one or more struts 612 may form cells 614 having different shapes. For example, the struts 612 may be configured to form generally rectangular and/or diamond-shaped cells 614. While the struts 612 are shown having generally thick structures, the one or more struts 612 may have wire-like and/or generally thin forms. In some examples, the inner frame 606 and/or outer frame 608 may be configured to maintain a uniform structure and/or strut 612 pattern along a length of the frame 602.

The implant 600 may be configured for delivery and/or placement at an SVC and/or IVC of a heart. For example, the implant 600 may be configured for placement at or near an in-flow junction of the SVC and/or IVC to the right atrium of the heart.

In some examples, the inner frame 606 may comprise alternating elongate arms, which can include upward-extending arms 617 and/or downward-extending arms 627. For example, the upward-extending arms 617 may be configured to extend from at or near a bulb 613 of the implant 600 and/or towards the distal end 618 of the implant 600. The one or more downward-extending arms 627 may be configured to extend from at or near the distal end 618 of the implant 600 and/or towards the proximal end 616 of the implant. In some examples, the one or more upward-extending arms 617 may comprise free ends disposed distally (e.g., nearer to the distal end 618) to corresponding free ends of the downward-extending arms 627.

The one or more upward-extending arms 617 and one or more downward-extending arms 627 may be disposed in an alternating manner about a circumference of the implant 600. For example, each upward-extending arm 617 may be disposed between two adjacent downward-extending arms 627 and/or each downward-extending arm 627 may be disposed between two adjacent upward-extending arms 617.

The upward-extending arms 617 and/or downward-extending arms 627 may be configured to form a cylindrical and/or circular basket and/or docking station for one or more prosthetic valves 607, as shown in FIG. 6B. In some examples, the alternating positioning of the upward-extending arms 617 and/or downward-extending arms 627 can advantageously distribute a load of the prosthetic valve 607 across the upward-extending arms 617 and/or downward-extending arms 627. For example, the free ends of the upward-extending arms 617 and/or downward-extending arms 627 may have be extended further towards a central axis of the implant 600 than other portions of the upward-extending arms 617 and/or downward-extending arms 627 and/or the free ends may bear an increased load of the prosthetic valve 607 relative to other portions of the upward-extending arms 617 and/or downward-extending arms 627. Accordingly, by alternating the upward-extending arms 617 and/or downward-extending arms 627, the load of the prosthetic valve 607 may be more evenly distributed along a length of the inner frame 606. The upward-extending arms 617 and/or downward-extending arms 627 may be configured to extend towards an axial center of the implant 600 and/or may have generally curved forms. The prosthetic valve 607 may comprise one or more leaflets 644.

In some examples, the frame 602 may first be delivered and/or deployed to a target anatomical location (e.g., a valve) and/or the prosthetic valve 607 may subsequently be deployed at and/or within the frame 602. Additionally or alternatively, the frame 602 and valve 607 may be deployed simultaneously and/or during a single procedure.

The implant 600 may be configured to prevent blockage of one or more branching blood vessels (e.g., hepatic veins) when implanted within a main blood vessel (e.g., the IVC). For example, the implant 600 may be configured to allow blood flow through the frame 602 of the implant 600 between the proximal end 616 and the inner frame 606.

The implant 600 may comprise one or more flared ends and/or arms at the distal end 618 and/or proximal end 616. In some examples, the frame 602 of the implant 600 may be laser-cut and/or may comprise one or more shape memory alloys (e.g., Nitinol). The frame 602 may comprise a network of struts 612 forming one or more cells 614. In some examples, the struts 612 may be interconnected and/or may form diamond-shaped and/or similarly shaped cells 614. The frame 602 may not comprise longitudinal-extending struts 612 (e.g., struts 612 extending in a generally straight line from the proximal end 616 to the distal end 618) and/or various struts 612 may be bent inwardly and/or outwardly to form the upward-extending arms 617 and/or downward-extending arms and/or to form one or more outward-extending anchors.

The implant 600 may comprise a sealing element 604 (e.g., skirt) extending at least partially along an inner and/or outer surface of the frame 602 and/or at least partially enclosing the frame 602. In some examples, the sealing element 604 may extend from the flared distal end 618 to the inner frame 606.

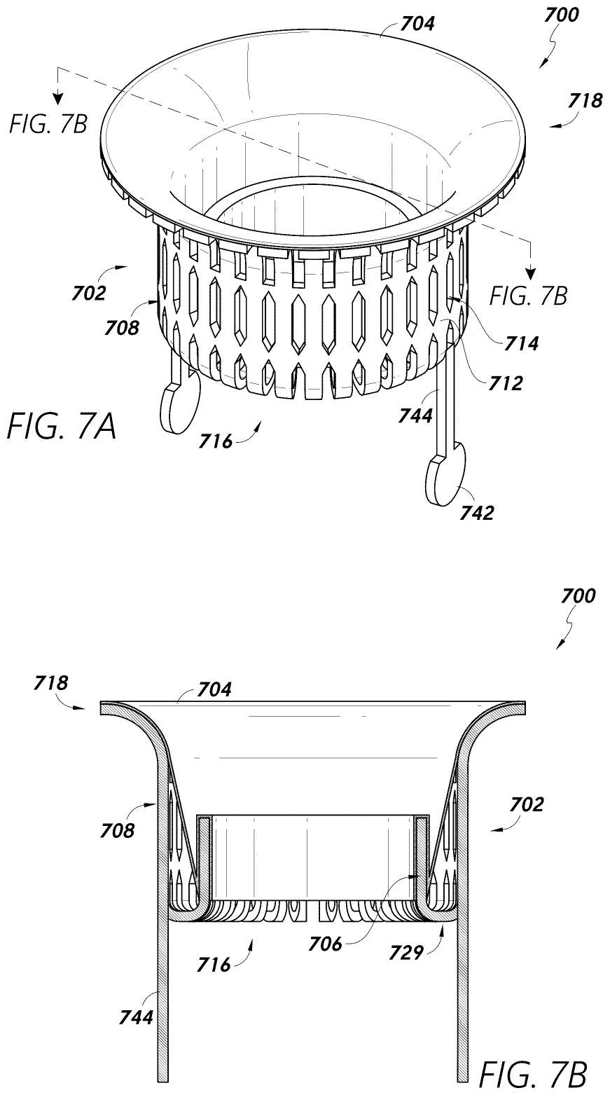

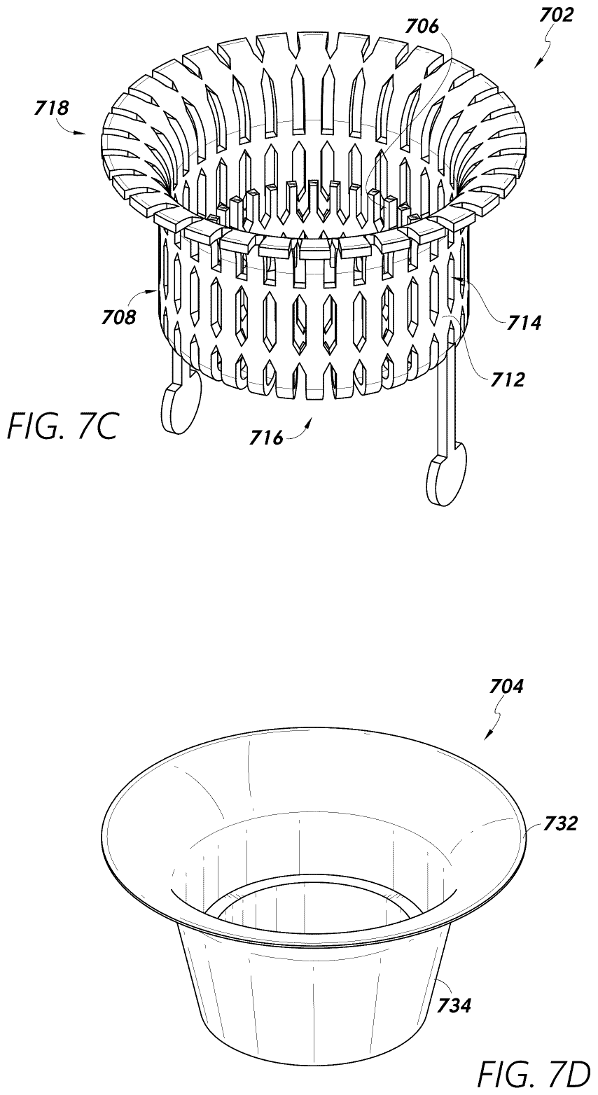

FIGS. 7A-7D illustrate components of an example implant 700 configured to dock and/or support one or more prosthetic valves and/or valve components in accordance with one or more instances. The implant 700 may comprise a frame 702 and/or a sealing element 704, which can include a skirt, covering, and/or similar device. FIG. 7A provides a perspective view of the implant 700. FIG. 7B provides a cross-sectional side view of the implant 700. FIG. 7C provides a perspective view of the frame 702 of the implant 700. FIG. 7D provides a perspective view of the sealing element 704 of the implant 700.

The frame 702 may be configured to form an inner frame 706 and/or an outer frame 708. The inner frame 706 may form a first diameter that is less than a second diameter of the outer frame 708. The inner frame 706 and outer frame 708 may be extensions of a common device and/or may extend from each other.

In some examples, the outer frame 708 may have a generally cylindrical form and/or may at least partially enclose a complete circumference of the inner frame 706 and/or of a portion of the sealing element 704. The outer frame 708 may comprise a network of struts 712, which can include wires, arms, bars, cords, walls, and/or similar components forming one or more cells 714 and/or openings through the outer frame 708. The one or more cells 714 may be configured to allow blood flow through the outer frame 708. The cells 714 may have any suitable shape and/or size. In the example shown in FIGS. 7A and 7B, the outer frame 708 may form generally elongate cells 714 extending approximately an entire length of the outer frame 708 and/or from a proximal end 716 to a distal end 718 of the implant 700. The one or more cells 714 may have triangular forms at end points of the one or more cells 714, as shown in FIGS. 7A and 7B. However, the one or more struts 712 may form cells 714 having different shapes. For example, the struts 712 may be configured to form generally rectangular and/or diamond-shaped cells 714. While the struts 712 are shown having generally thick structures, the one or more struts 712 may have wire-like and/or generally thin forms. In some examples, the inner frame 706 and/or outer frame 708 may be configured to maintain a uniform structure and/or strut 712 pattern along a length of the frame 702.

The implant 700 may be configured for delivery and/or placement at an SVC and/or IVC of a heart. For example, the implant 700 may be configured for placement at or near an in-flow junction of the SVC and/or IVC to the right atrium of the heart.

In some examples, the implant 700 may have a relatively shorter (e.g., between the proximal end 716 and the distal end 718 of the implant 700) form relative to other caval implants. The frame 702 may be at least partially composed of one or more shape memory alloys (e.g., Nitinol) and/or may be laser-cut into a desired form. In some examples, the frame 702 and/or sealing element 704 may have a generally flared form at or near the distal end 718 of the implant 700. For example, the distal end 718 of the implant 700 may be flared and/or conical in shape to extend outward into the right atrium and/or to form a grip onto a rim of the right atrium. In some examples, the sealing element 704 may be configured to extend beyond the frame 702 and/or may form an extension of the frame 702 to extend further laterally and/or longitudinally relative to the frame 702. In some examples, the outer frame 708 may be at least partially flared at or near the distal end 718 of the implant 700. The inner frame 706 may have a generally straight form extending towards the distal end 718 and/or may flare outwardly towards the outer frame 708 at or near the proximal end 716.

The proximal end 716 may have a straight or flared form. In the example shown in FIGS. 7A and 7B, the inner frame 706 and outer frame 708 may join together at or near the proximal end 716 and/or may flare outwardly to improve a proximal seal of the implant 700. Alternatively, the frame 702 may be configured to flare inwardly at or near the proximal end 716 and/or may be configured to taper inwardly to approximate a shaped of a blood vessel. For example, the outer frame 708 may have a generally tapered and/or inwardly angled form extending from the distal end 718 to the proximal end 716.

The inner frame 706 may be configured to extend outwardly and/or towards the proximal end 716. The outer frame 708 and inner frame 706 may form a V-shaped juncture at or near the proximal end 716. The implant 700 may form a flared end at the distal end 718 and/or proximal end 716 to engage an atrium and/or other anatomy when implanted. The sealing element 704 may be configured to extend from the flared distal end 718 and/or may extend at least partially along an inner and/or outer surface of the inner frame 706. In some examples, the sealing element 704 may be configured to bridge a gap and/or space between the inner frame 706 and the outer frame 708 at or near the distal end 718 of the implant 700.

The implant 700 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 704 may not extend along a complete length of the frame 702. For example, the sealing element 704 may extend from the distal end 718 of the implant 700 to a midsection and/or midpoint of the implant 700 and/or inner frame 706. In some examples, the sealing element 704 may not extend to a junction between the inner frame 706 and outer frame 708 at or near the proximal end 716 of the implant 700. Moreover, the sealing element 704 may not extend over an outwardly extending portion of the inner frame 706 and/or may only extend along a generally straight segment of the inner frame 706. The implant 700 may be configured such that, when implanted at the junction between the IVC and the right atrium, the sealing element 704 may not extend over the one or more hepatic veins and/or branching blood vessels. In some examples, the frame 702 may at least partially extend over the hepatic veins and/or branching blood vessels and/or may allow blood flow from the branching blood vessels through cells 714 of the frame 702. The inner frame 706 may be configured to hold the sealing element 704 distally from the outer frame 708 and/or walls of the blood vessel such that the sealing element 704 may not create blockage of one or more branching blood vessels if the sealing element 704 does extend across one or more branching blood vessels.

In some examples, the implant 700 may comprise one or more radiopaque markers configured to facilitate delivery and/or use of the implant 700. For example, one or more markers may be disposed along the inner frame 706 (e.g., at a midsection of the inner frame 706 and/or at a point at which the sealing element 704 ends) to assist with aligning and/or docking one or more prosthetic valves at the implant 700 and/or inner frame 706. In another example, the implant 700 may comprise one or more markers at the outer frame 708 and/or sealing element 704 at or near the distal end 718 to facilitate positioning of the implant 700 and/or distal end 718 at the atrium junction and/or other anatomy.

In some examples, the frame 702 and/or outer frame 708 may comprise one or more elongate arms and/or fingers configured to flare out at the distal end 718. The one or more arms and/or fingers may be configured to support a flared end of the sealing element 704 and/or to approximate a curvature of the junction between the blood vessel and right atrium and/or other chamber. The sealing element 704 may be composed of any suitable material, which can include fabric (e.g., polyester), cloth, and/or rubber. The one or more arms and/or fingers may be configured extend under the sealing element 704 to hold the sealing element 704 up and/or otherwise support the sealing element 704. In some examples, the sealing element 704 may be configured to extend over and/or under the frame 702 and/or arms at the distal end 718. For example, the sealing element 704 may be configured to wrap around the frame 702 and/or extend between the frame 702 at the distal end 718 and the native anatomy to improve sealing of the frame 702 with the anatomy.

The frame may be coupled to the sealing element 704 and/or may be configured to mate with the sealing element 704. In some examples, one or more sutures and/or other attachment features (e.g., clips, hooks, notches, stitches, adhesives, etc.) may be used to form an attachment between the frame 702 and the sealing element 704.

In some examples, the implant 700 may be configured to cause native anatomy to conform to a size and/or shape of the implant 700. For example, the outer frame 708 and/or distal end 718 of the implant 700 may be configured to press outwardly against the native tissue to create a larger opening to support a prosthetic valve. In some examples, the implant 700 may comprise one or more bulbs configured to press outwardly against walls of a blood vessel and/or may be configured to correspondingly press walls of the blood vessel outwardly.

The outer frame 708 and/or inner frame 706 may be configured to form relatively large cells 714. For example, a majority of a surface area of the outer frame 708 and/or inner frame 706 may be empty space between struts 712 of the outer frame 708 and/or inner frame 706.

The sealing element 704 (e.g., skirt) may have any suitable size and/or shape. In some examples, the sealing element 704 can comprise a proximal portion 732 having a smaller width and/or diameter than a distal portion 734 of the sealing element 704. The proximal portion 732 may have a tubular and/or cylindrical form. The distal portion 734 may flare out from the proximal portion 732 and/or may gradually extend into a larger diameter to provide a distal sealing end of the implant 700.

In some examples, the frame 702 of the implant 700 may be laser-cut and/or may comprise one or more shape memory alloys (e.g., Nitinol). The frame 702 may comprise a network of struts 712 forming one or more cells 714. In some examples, the struts 712 may be interconnected and/or may form emerald-shaped (e.g., elongated diamond shapes) and/or similarly shaped cells 714.

The outer frame 708 may extend into the inner frame 706. In some examples, a curved section 729 of the frame 702 may curve the outer frame 708 inward and/or upward to form the inner frame 706. The curved section 729 may be turned upward and inward such that the inner frame 706 forms an internal diameter that is less than an external diameter of the outer frame 708. In some examples, the inner frame 706 and/or curved section 729 may comprise an equal and/or similar design of struts 712 and/or cells 714 as the outer frame 708. The inner frame 706 may extend upwardly towards the distal end 718 and/or may form a free end and/or a free cylindrical and/or tubular shape disposed at least partially and/or entirely within the outer frame 708. The curved section 729 may from a U-shaped juncture between the inner frame 706 and the outer frame 708.

The distal end 718 may have a generally flared form and/or may be configured to engage an opening of a blood vessel (e.g., the IVC) into a chamber (e.g., the atrium) when implanted within the blood vessel. In some examples, the implant 700 may comprise a sealing element (e.g., skirt). The outer frame 708 may extend from the distal end 718 to the proximal end 716 and/or the inner frame 706 may represent a change in direction of the frame 702 and/or may extend towards the distal end 718. The sealing element 704 may be configured to extend from the flared distal end 718 to the curved section 729 and/or at least partially along the inner frame 706 and/or may be configured to engage and/or dock a prosthetic valve (not shown). In some examples, the sealing element 704 may not extend along an entire length of the frame 702 (e.g., may not extend the full length of the outer frame 708) and/or may not be configured to cover one or more branching blood vessels (e.g., hepatic veins). The outer frame 708 and/or inner frame 706 may comprise a generally open mesh of struts 712 forming open cells 714 configured to allow blood flow through the frame 702.

In some examples, the frame 702 may comprise an outward-extending bulb at or near the proximal end 716 of the implant 700. The bulb may be configured to facilitate anchoring of the implant 700 with the surrounding anatomy.

The implant 700 may comprise one or more anchors 742 disposed at distal ends of one or more anchoring arms 744 extending from the proximal end 716 of the implant 700. In some examples, the anchors 742 may be configured for anchoring to one or more catheters and/or other delivery devices.

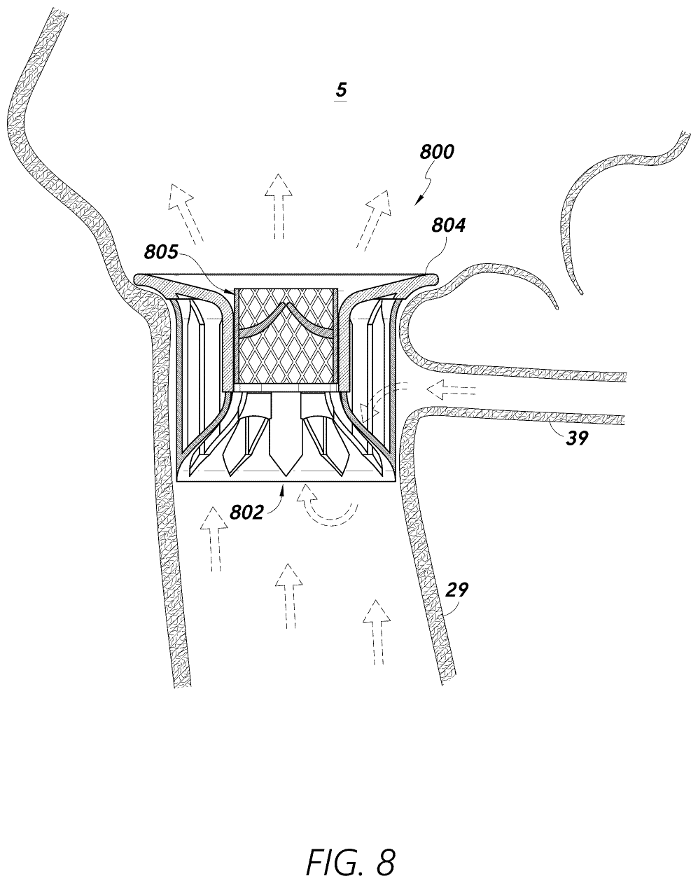

FIG. 8 illustrates components of an example implant 800 implanted within a blood vessel 29 (e.g., the IVC) and/or configured to dock one or more prosthetic valves 805 and/or valve components in accordance with one or more instances. The implant 800 may comprise a frame 802 and/or a sealing element 804, which can include a skirt, covering, and/or similar device.

The frame 802 may be configured to form an inner frame and/or an outer frame. The inner frame may form a first diameter that is less than a second diameter of the outer frame. The inner frame and outer frame may be extensions of a common device and/or may extend from each other.

In some examples, the outer frame may have a generally cylindrical form and/or may at least partially enclose a complete circumference of the inner frame and/or of a portion of the sealing element 804. The outer frame may comprise a network of strut, which can include wires, arms, bars, cords, walls, and/or similar components forming one or more cells 814 and/or openings through the outer frame. The one or more cells 814 may be configured to allow blood flow through the outer frame. The cells 814 may have any suitable shape and/or size. In the example shown in FIGS. 8A and 8B, the outer frame may form generally elongate cells 814 extending approximately an entire length of the outer frame 808 and/or from a proximal end to a distal end of the implant 800. The one or more cells 814 may have triangular forms at end points of the one or more cells 814, as shown in FIGS. 8A and 8B. However, the one or more struts may form cells 814 having different shapes. For example, the strut may be configured to form generally rectangular and/or diamond-shaped cells 814. While the struts are shown having generally thick structures, the one or more struts may have wire-like and/or generally thin forms. In some examples, the inner frame and/or outer frame may be configured to maintain a uniform structure and/or strut pattern along a length of the frame 802.

The implant 800 may be configured for delivery and/or placement at an SVC and/or IVC of a heart. For example, the implant 800 may be configured for placement at or near an in-flow junction of the SVC and/or IVC to the right atrium of the heart.

In some examples, the implant 800 may have a relatively shorter (e.g., between the proximal end and the distal end of the implant 800) form relative to other caval implants. The frame 802 may be at least partially composed of one or more shape memory alloys (e.g., Nitinol) and/or may be laser-cut into a desired form. In some examples, the frame 802 and/or sealing element 804 may have a generally flared form at or near the distal end of the implant 800. For example, the distal end of the implant 800 may be flared and/or conical in shape to extend outward into the right atrium and/or to form a grip onto a rim of the right atrium. In some examples, the sealing element 804 may be configured to extend beyond the frame 802 and/or may form an extension of the frame 802 to extend further laterally and/or longitudinally relative to the frame 802. In some examples, the outer frame may be at least partially flared at or near the distal end of the implant 800. The inner frame may have a generally straight form extending towards the distal end and/or may flare outwardly towards the outer frame at or near the proximal end.

The proximal end may have a straight or flared form. In the example shown in FIGS. 8A and 8B, the inner frame and outer frame may join together at or near the proximal end and/or may flare outwardly to improve a proximal seal of the implant 800. Alternatively, the frame 802 may be configured to flare inwardly at or near the proximal end and/or may be configured to taper inwardly to approximate a shaped of a blood vessel 29. For example, the outer frame may have a generally tapered and/or inwardly angled form extending from the distal end to the proximal end.

In some examples, the inner frame may comprise a network of strut forming one or more cells 814, similarly to the outer frame. The inner frame may comprise one or more elongate arms, prongs, fingers, and/or similar device extending from the frame 802 at or near the proximal end and/or a midsection of the frame 802. The one or more arms may be configured to join together to form a generally circular, tubular, and/or cylindrical midsection of the frame 802. A prosthetic valve 805 and/or the sealing element 804 may be configured to be mounted to the midsection. In some examples, the one or more arms may not be interconnected and/or may comprise free ends configured to support the sealing element 804. Alternatively, the one or more arms may be configured to flare outwardly at or near the distal end of the implant 800 and/or to join the outer frame at or near the distal end.

The inner frame may be configured to extend outwardly and/or towards the proximal end. The outer frame and inner frame may form a V-shaped juncture at or near the proximal end. The implant 800 may form a flared end at the distal end and/or proximal end to engage an atrium and/or other anatomy when implanted. The sealing element 804 may be configured to extend from the flared distal end and/or may extend at least partially along an inner and/or outer surface of the inner frame. In some examples, the sealing element 804 may be configured to bridge a gap and/or space between the inner frame and the outer frame at or near the distal end of the implant 800.

The implant 800 may be configured to present minimal blockage of one or more hepatic veins 39 and/or other branching blood vessels. For example, a heart may generally include one or more hepatic veins branching into the IVC at or near a junction between the IVC and the right atrium. In some examples, the sealing element 804 may not extend along a complete length of the frame 802. For example, the sealing element 804 may extend from the distal end of the implant 800 to a midsection and/or midpoint of the implant 800 and/or inner frame. In some examples, the sealing element 804 may not extend to a junction between the inner frame and outer frame at or near the proximal end of the implant 800. Moreover, the sealing element 804 may not extend over an outwardly extending portion of the inner frame and/or may only extend along a generally straight segment of the inner frame. The implant 800 may be configured such that, when implanted at the junction between the IVC and the right atrium, the sealing element 804 may not extend over the one or more hepatic veins 39 and/or branching blood vessels. In some examples, the frame 802 may at least partially extend over the hepatic veins 39 and/or branching blood vessels and/or may allow blood flow from the branching blood vessels 39 through cells 814 of the frame 802. The inner frame may be configured to hold the sealing element 804 distally from the outer frame and/or walls of the blood vessel 29 such that the sealing element 804 may not create blockage of one or more branching blood vessels 39 if the sealing element 804 does extend across one or more branching blood vessels 39.

In some examples, the implant 800 may comprise one or more radiopaque markers configured to facilitate delivery and/or use of the implant 800. For example, one or more markers may be disposed along the inner frame (e.g., at a midsection of the inner frame and/or at a point at which the sealing element 804 ends) to assist with aligning and/or docking one or more prosthetic valves 805 at the implant 800 and/or inner frame. In another example, the implant 800 may comprise one or more markers at the outer frame and/or sealing element 804 at or near the distal end to facilitate positioning of the implant 800 and/or distal end at the atrium junction and/or other anatomy.

In some examples, the frame 802 and/or outer frame may comprise one or more elongate arms and/or fingers configured to flare out at the distal end. The one or more arms and/or fingers may be configured to support a flared end of the sealing element 804 and/or to approximate a curvature of the junction between the blood vessel 29 and right atrium 5 and/or other chamber. The sealing element 804 may be composed of any suitable material, which can include fabric (e.g., polyester), cloth, and/or rubber. The one or more arms and/or fingers may be configured extend under the sealing element 804 to hold the sealing element 804 up and/or otherwise support the sealing element 804. In some examples, the sealing element 804 may be configured to extend over and/or under the frame 802 and/or arms at the distal end. For example, the sealing element 804 may be configured to wrap around the frame 802 and/or extend between the frame 802 at the distal end and the native anatomy to improve sealing of the frame 802 with the anatomy.

The frame 802 may be coupled to the sealing element 804 and/or may be configured to mate with the sealing element 804. In some examples, one or more sutures and/or other attachment features (e.g., clips, hooks, notches, stitches, adhesives, etc.) may be used to form an attachment between the frame 802 and the sealing element 804.

In some examples, the implant 800 may be configured to cause native anatomy to conform to a size and/or shape of the implant 800. For example, the outer frame and/or distal end of the implant 800 may be configured to press outwardly against the native tissue to create a larger opening to support a prosthetic valve 805. In some examples, the implant 800 may comprise one or more bulbs configured to press outwardly against walls of a blood vessel 29 and/or may be configured to correspondingly press walls of the blood vessel 29 outwardly.

The outer frame and/or inner frame may be configured to form relatively large cells 814. For example, a majority of a surface area of the outer frame and/or inner frame may be empty space between strut of the outer frame and/or inner frame.

In some examples, the outer frame may have generally linear and/or straight sides. For example, a proximal end of the implant 800 (e.g., below one or more hepatic veins) may not be flared and/or may not increase in diameter. A distal end of the implant 800 (e.g., extending into the right atrium 5 and/or other chamber) may be generally flared to anchor the implant 800 at or near a junction between the right atrium 5 and the blood vessel 29.

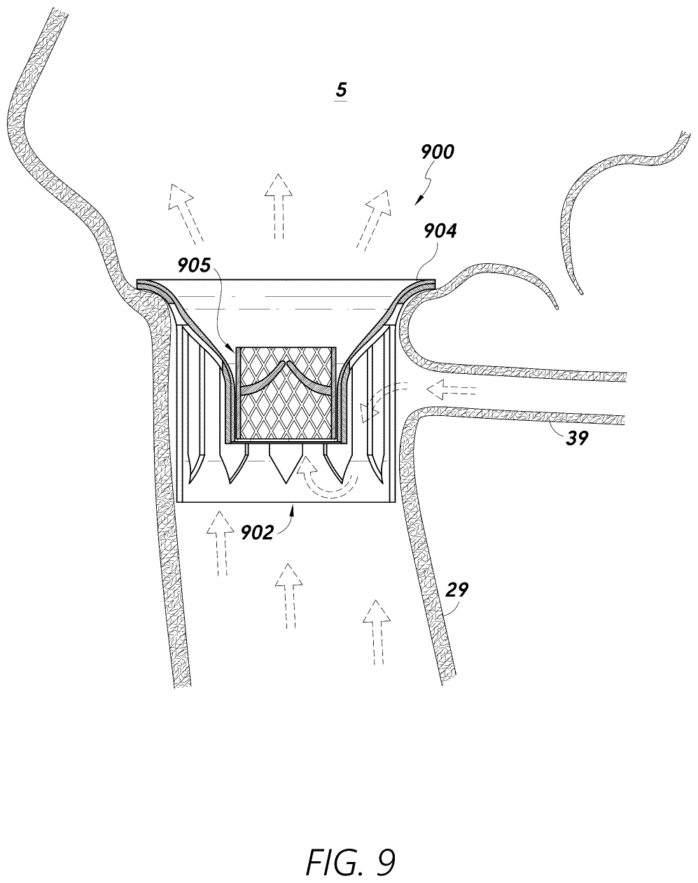

FIG. 9 illustrate components of an example implant 900 implanted within a blood vessel 29 (e.g., the IVC) and/or configured to dock one or more prosthetic valves 905 and/or valve components in accordance with one or more instances. The implant 900 may comprise a frame 902 and/or a sealing element 904, which can include a skirt, covering, and/or similar device.

The frame 902 may be configured to form an inner frame and/or an outer frame. The inner frame may form a first diameter that is less than a second diameter of the outer frame. The inner frame and outer frame may be extensions of a common device and/or may extend from each other.

In some examples, the outer frame may have a generally cylindrical form and/or may at least partially enclose a complete circumference of the inner frame and/or of a portion of the sealing element 904. The outer frame may comprise a network of struts, which can include wires, arms, bars, cords, walls, and/or similar components forming one or more cells 914 and/or openings through the outer frame. The one or more cells 914 may be configured to allow blood flow through the outer frame. The cells 914 may have any suitable shape and/or size. In the example shown in FIGS. 9A and 9B, the outer frame may form generally elongate cells 914 extending approximately an entire length of the outer frame 908 and/or from a proximal end to a distal end of the implant 900. The one or more cells 914 may have triangular forms at end points of the one or more cells 914, as shown in FIGS. 9A and 9B. However, the one or more struts may form cells 914 having different shapes. For example, the struts may be configured to form generally rectangular and/or diamond-shaped cells 914. While the struts are shown having generally thick structures, the one or more struts may have wire-like and/or generally thin forms. In some examples, the inner frame and/or outer frame may be configured to maintain a uniform structure and/or strut pattern along a length of the frame 902.

The implant 900 may be configured for delivery and/or placement at an SVC and/or IVC of a heart. For example, the implant 900 may be configured for placement at or near an in-flow junction of the SVC and/or IVC to the right atrium of the heart.

In some examples, the implant 900 may have a relatively shorter (e.g., between the proximal end and the distal end of the implant 900) form relative to other caval implants. The frame 902 may be at least partially composed of one or more shape memory alloys (e.g., Nitinol) and/or may be laser-cut into a desired form. In some examples, the frame 902 and/or sealing element 904 may have a generally flared form at or near the distal end of the implant 900. For example, the distal end of the implant 900 may be flared and/or conical in shape to extend outward into the right atrium and/or to form a grip onto a rim of the right atrium. In some examples, the sealing element 904 may be configured to extend beyond the frame 902 and/or may form an extension of the frame 902 to extend further laterally and/or longitudinally relative to the frame 902. In some examples, the outer frame may be at least partially flared at or near the distal end of the implant 900. The inner frame may have a generally straight form extending towards the distal end and/or may flare outwardly towards the outer frame at or near the proximal end.

The proximal end may have a straight or flared form. In the example shown in FIGS. 9A and 9B, the inner frame and outer frame may join together at or near the proximal end and/or may flare outwardly to improve a proximal seal of the implant 900. Alternatively, the frame 902 may be configured to flare inwardly at or near the proximal end and/or may be configured to taper inwardly to approximate a shaped of a blood vessel 29. For example, the outer frame may have a generally tapered and/or inwardly angled form extending from the distal end to the proximal end.

In some examples, the inner frame may comprise a network of struts forming one or more cells 914, similarly to the outer frame. The inner frame may comprise one or more elongate arms, prongs, fingers, and/or similar device extending from the frame 902 at or near the proximal end and/or a midsection of the frame 902. The one or more arms may be configured to join together to form a generally circular, tubular, and/or cylindrical midsection of the frame 902. A prosthetic valve 905 and/or the sealing element 904 may be configured to be mounted to the midsection. In some examples, the one or more arms may not be interconnected and/or may comprise free ends configured to support the sealing element 904. Alternatively, the one or more arms may be configured to flare outwardly at or near the distal end of the implant 900 and/or to join the outer frame at or near the distal end.

The inner frame may be configured to extend outwardly and/or towards the proximal end. The outer frame and inner frame may form a V-shaped juncture at or near the proximal end. The implant 900 may form a flared end at the distal end and/or proximal end to engage an atrium and/or other anatomy when implanted. The sealing element 904 may be configured to extend from the flared distal end and/or may extend at least partially along an inner and/or outer surface of the inner frame. In some examples, the sealing element 904 may be configured to bridge a gap and/or space between the inner frame and the outer frame at or near the distal end of the implant 900.

The implant 900 may be configured to present minimal blockage of one or more hepatic veins 39 and/or other branching blood vessels. For example, a heart may generally include one or more hepatic veins branching into the IVC at or near a junction between the IVC and the right atrium. In some examples, the sealing element 904 may not extend along a complete length of the frame 902. For example, the sealing element 904 may extend from the distal end of the implant 900 to a midsection and/or midpoint of the implant 900 and/or inner frame. In some examples, the sealing element 904 may not extend to a junction between the inner frame and outer frame at or near the proximal end of the implant 900. Moreover, the sealing element 904 may not extend over an outwardly extending portion of the inner frame and/or may only extend along a generally straight segment of the inner frame. The implant 900 may be configured such that, when implanted at the junction between the IVC and the right atrium, the sealing element 904 may not extend over the one or more hepatic veins 39 and/or branching blood vessels. In some examples, the frame 902 may at least partially extend over the hepatic veins 39 and/or branching blood vessels and/or may allow blood flow from the branching blood vessels 39 through cells 914 of the frame 902. The inner frame may be configured to hold the sealing element 904 distally from the outer frame and/or walls of the blood vessel 29 such that the sealing element 904 may not create blockage of one or more branching blood vessels 39 if the sealing element 904 does extend across one or more branching blood vessels 39.

In some examples, the implant 900 may comprise one or more radiopaque markers configured to facilitate delivery and/or use of the implant 900. For example, one or more markers may be disposed along the inner frame (e.g., at a midsection of the inner frame and/or at a point at which the sealing element 904 ends) to assist with aligning and/or docking one or more prosthetic valves 905 at the implant 900 and/or inner frame. In another example, the implant 900 may comprise one or more markers at the outer frame and/or sealing element 904 at or near the distal end to facilitate positioning of the implant 900 and/or distal end at the atrium junction and/or other anatomy.

In some examples, the frame 902 and/or outer frame may comprise one or more elongate arms and/or fingers configured to flare out at the distal end. The one or more arms and/or fingers may be configured to support a flared end of the sealing element 904 and/or to approximate a curvature of the junction between the blood vessel 29 and right atrium 9 and/or other chamber. The sealing element 904 may be composed of any suitable material, which can include fabric (e.g., polyester), cloth, and/or rubber. The one or more arms and/or fingers may be configured extend under the sealing element 904 to hold the sealing element 904 up and/or otherwise support the sealing element 904. In some examples, the sealing element 904 may be configured to extend over and/or under the frame 902 and/or arms at the distal end. For example, the sealing element 904 may be configured to wrap around the frame 902 and/or extend between the frame 902 at the distal end and the native anatomy to improve sealing of the frame 902 with the anatomy.

The frame 902 may be coupled to the sealing element 904 and/or may be configured to mate with the sealing element 904. In some examples, one or more sutures and/or other attachment features (e.g., clips, hooks, notches, stitches, adhesives, etc.) may be used to form an attachment between the frame 902 and the sealing element 904.

In some examples, the implant 900 may be configured to cause native anatomy to conform to a size and/or shape of the implant 900. For example, the outer frame and/or distal end of the implant 900 may be configured to press outwardly against the native tissue to create a larger opening to support a prosthetic valve 905. In some examples, the implant 900 may comprise one or more bulbs configured to press outwardly against walls of a blood vessel 29 and/or may be configured to correspondingly press walls of the blood vessel 29 outwardly.

The outer frame and/or inner frame may be configured to form relatively large cells 914. For example, a majority of a surface area of the outer frame and/or inner frame may be empty space between struts of the outer frame and/or inner frame.

The sealing element 904 (e.g., skirt) may have any suitable size and/or shape. In some examples, the sealing element 904 can comprise a proximal portion having a smaller width and/or diameter than a distal portion of the sealing element 904. The proximal portion may have a tubular and/or cylindrical form. The distal portion may flare out from the proximal portion and/or may gradually extend into a larger diameter to provide a distal sealing end of the implant 900.

In some examples, the outer frame may have generally linear and/or straight sides. For example, a proximal end of the implant 900 (e.g., below one or more hepatic veins) may not be flared and/or may not increase in diameter. A distal end of the implant 900 (e.g., extending into the right atrium 9 and/or other chamber) may be generally flared to anchor the implant 900 at or near a junction between the right atrium 9 and the blood vessel 29.

At a distal end of the implant 900, the outer frame and inner frame may join together and/or the inner frame may extend away from the outer frame and/or along an inner lumen formed by the outer frame. In some examples, the outer frame and inner frame may both have a generally flared and/or conical form at or near the distal end of the implant 900. The inner frame may have a flaring angle that is greater than the outer frame such that the diameter of the inner frame may be less than the diameter of the outer frame. The inner frame may be configured to extend along at least a portion of the length of the outer frame.

The flared end (e.g., distal end) of the outer frame and/or inner frame may be configured to engage an atrium and/or other chamber when implanted. The sealing element 904 may be configured to extend along the inner frame and/or may be configured to wrap around the outer frame and/or extend between the outer frame and the native tissue. The outer frame may be configured to extend into the IVC and/or other blood vessel and/or the sealing element 904 may be configured to extend from the outer frame to an inner surface of the inner frame. The sealing element 904 may be configured for engagement with a prosthetic valve and/or other implant. For example, the sealing element 904 may be configured to provide a mounting surface for a prosthetic valve and/or may be configured to securely hold the prosthetic valve. The sealing element 904 may be configured to extend along only a portion of the implant 900 such that the sealing element 904 may not extend across one or more branching vessels of the blood vessel.

In some examples, the implant 900 may comprise one or more outward bulbs configured to extend outwardly from the diameter of the outer frame to facilitate anchoring of the implant 900 within a blood vessel.

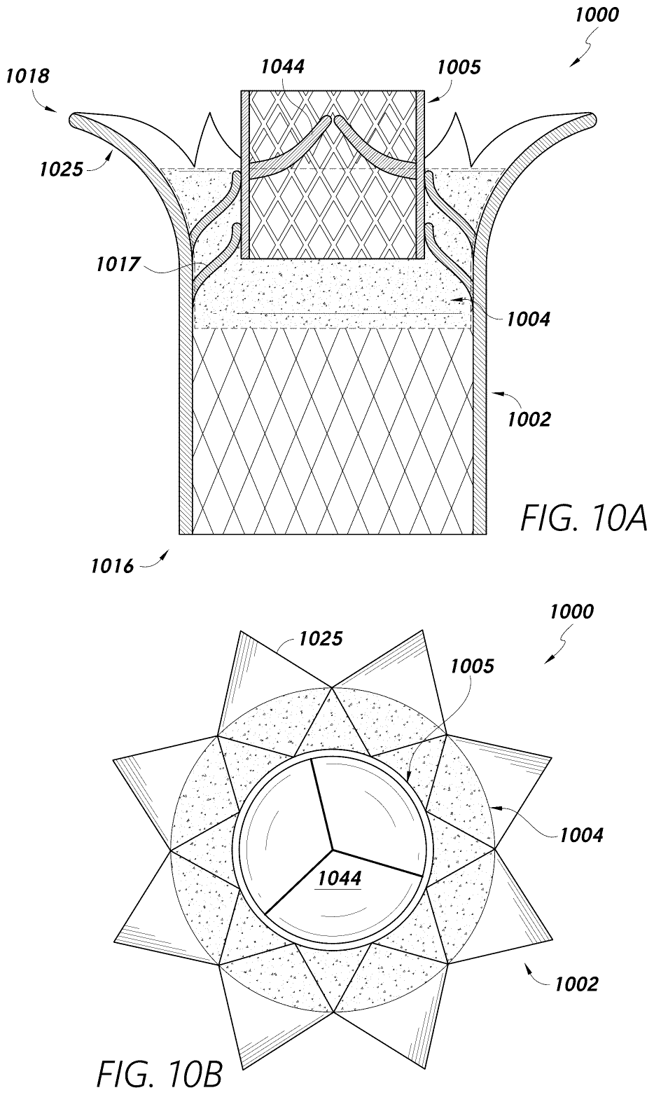

FIGS. 10A and 10B illustrate components of an example implant 1000 configured to dock and/or support one or more prosthetic valves and/or valve components in accordance with one or more instances. The implant 1000 may comprise a frame 1002 and/or a sealing element 1004, which can include a skirt, covering, and/or similar device. FIG. 10A provides a cross-sectional side view of the implant 1000. FIG. 10C provides an overhead view of the implant 1000.

In some examples, the frame 1002 may have a generally cylindrical form and/or may at least partially enclose a complete circumference of the implant 1000 and/or of a portion of the sealing element 1004. The frame 1002 may comprise a network of struts, which can include wires, arms, bars, cords, walls, and/or similar components forming one or more cells 1014 and/or openings through the frame 1002. The one or more cells 1014 may be configured to allow blood flow through the frame 1002. The cells 1014 may have any suitable shape and/or size. In the example shown in FIGS. 10A and 10B, the frame 1002 may form generally elongate cells 1014 extending approximately an entire length of the outer frame 1008 and/or from a proximal end 1016 to a distal end 1018 of the implant 1000. The one or more cells 1014 may have triangular forms at end points of the one or more cells 1014, as shown in FIGS. 10A and 10B. However, the one or more struts may form cells 1014 having different shapes. For example, the struts may be configured to form generally rectangular and/or diamond-shaped cells 1014. While the struts are shown having generally thick structures, the one or more struts may have wire-like and/or generally thin forms. In some examples, the frame 1002 may be configured to maintain a uniform structure and/or strut pattern along a length of the frame 1002.

The implant 1000 may be configured for delivery and/or placement at an SVC and/or IVC of a heart. For example, the implant 1000 may be configured for placement at or near an in-flow junction of the SVC and/or IVC to the right atrium of the heart.

In some examples, the implant 1000 may have a relatively short (e.g., between the proximal end 1016 and the distal end 1018 of the implant 1000) form relative to other caval implants. The frame 1002 may be at least partially composed of one or more shape memory alloys (e.g., Nitinol) and/or may be laser-cut into a desired form. In some examples, the frame 1002 and/or sealing element 1004 may have a generally flared form at or near the distal end 1018 of the implant 1000. For example, the distal end 1018 of the implant 1000 may be flared and/or conical in shape to extend outward into the right atrium and/or to form a grip onto a rim of the right atrium. In some examples, the sealing element 1004 may be configured to extend beyond the frame 1002 and/or may form an extension of the frame 1002 to extend further laterally and/or longitudinally relative to the frame 1002. In some examples, the frame 1002 may be at least partially flared at or near the distal end 1018 of the implant 1000.

The proximal end 1016 may have a straight or flared form. Alternatively, the frame 1002 may be configured to flare inwardly at or near the proximal end 1016 and/or may be configured to taper inwardly to approximate a shaped of a blood vessel. For example, the frame 1002 may have a generally tapered and/or inwardly angled form extending from the distal end 1018 to the proximal end 1016.

The implant 1000 may comprise one or more arms 1017 extending upwardly (e.g., towards the distal end 1018 of the implant 1000) from the frame 1002. The one or more arms 1017 may have generally curved forms and/or may be configured to grasp and/or contact a prosthetic valve 1005 placed into the implant 1000. The prosthetic valve 1005 may comprise one or more leaflets 1044 extending across a central lumen of the valve 1005. In some examples, the one or more arms 1017 may be configured to be disposed around a circumference of an inner surface of the frame 1002.

In some examples, the sealing element 1004 may be configured to extend around an outer surface of the frame 1002 and/or along an inner surface of the frame 1002. The one or more arms 1017 may be configured to extend through the sealing element 1004 and/or the sealing element 1004 may be configured to extend along the one or more arms 1017.

The implant 1000 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 1004 may not extend along a complete length of the frame 1002. For example, the sealing element 1004 may extend from the distal end 1018 of the implant 1000 to a midsection and/or midpoint of the implant 1000 and/or frame 1002. In some examples, the sealing element 1004 may not extend beyond junction points between the one or more arms 1017 and the frame 1002. Moreover, the sealing element 1004 may not extend over an outwardly extending portion of the arms 1017 and/or may only extend along a generally straight segment of the frame 1002. The implant 1000 may be configured such that, when implanted at the junction between the IVC and the right atrium, the sealing element 1004 may not extend over the one or more hepatic veins and/or branching blood vessels. In some examples, the frame 1002 may at least partially extend over the hepatic veins and/or branching blood vessels and/or may allow blood flow from the branching blood vessels through cells 1014 of the frame 1002. The one or more arms 1017 may be configured to hold the sealing element 1004 distally from the frame 1002 and/or walls of the blood vessel such that the sealing element 1004 may not create blockage of one or more branching blood vessels if the sealing element 1004 does extend across one or more branching blood vessels.

In some examples, the implant 1000 may comprise one or more radiopaque markers configured to facilitate delivery and/or use of the implant 1000. For example, one or more markers may be disposed along the frame 1002 and/or arms 1017 to assist with aligning and/or docking one or more prosthetic valves 1005 at the implant 1000 and/or elongate arms 1017. In another example, the implant 1000 may comprise one or more markers at the frame 1002 and/or sealing element 1004 at or near the distal end 1018 to facilitate positioning of the implant 1000 and/or distal end 1018 at the atrium junction and/or other anatomy.

In some examples, the frame 1002 may comprise one or more elongate arms 1025 and/or fingers configured to flare out at the distal end 1018. The one or more arms 1025 and/or fingers may be configured to support a flared end of the sealing element 1004 and/or to approximate a curvature of the junction between the blood vessel and right atrium and/or other chamber. In some examples, the one or more arms 1025 may have generally triangular shapes and/or may form triangular extensions from the frame 1002.

The sealing element 1004 may be composed of any suitable material, which can include fabric (e.g., polyester), cloth, and/or rubber. The one or more arms 1025 and/or fingers may be configured extend under the sealing element 1004 to hold the sealing element 1004 up and/or otherwise support the sealing element 1004. In some examples, the sealing element 1004 may be configured to extend over and/or under the frame 1002 and/or arms at the distal end 1018. For example, the sealing element 1004 may be configured to wrap around the frame 1002 and/or extend between the frame 1002 at the distal end 1018 and the native anatomy to improve sealing of the frame 1002 with the anatomy.

The frame 1002 may be coupled to the sealing element 1004 and/or may be configured to mate with the sealing element 1004. In some examples, one or more sutures and/or other attachment features (e.g., clips, hooks, notches, stitches, adhesives, etc.) may be used to form an attachment between the frame 1002 and the sealing element 1004.

In some examples, the implant 1000 may be configured to cause native anatomy to conform to a size and/or shape of the implant 1000. For example, the frame 1002 and/or distal end 1018 of the implant 1000 may be configured to press outwardly against the native tissue to create a larger opening to support a prosthetic valve. In some examples, the implant 1000 may comprise one or more bulbs configured to press outwardly against walls of a blood vessel and/or may be configured to correspondingly press walls of the blood vessel outwardly.

The frame 1002 may be configured to form relatively large cells 1014. For example, a majority of a surface area of the frame 1002 may be empty space between struts of the frame 1002.

The arms 1017 of the implant 1000 may extend upwardly from a midsection of the frame 1002 and/or from the proximal end 1016 of the implant 1000 to and/or towards the distal end 1018. For example, the one or more arms 1017 may extend at an approximately 45-degree angle from the frame 1002 and/or may extend generally upwardly generally in parallel with the frame 1002 along a midsection of the implant 1000.

The implant 1000 may have a flared distal end 1018. The flared distal end 1018 may comprise a series of triangle- and/or diamond-shaped petals 1025. The flared distal end 1018 may also comprise one or more inward-extending arms 1017 (e.g., prongs, arms, spikes, etc.) configured to grasp, pierce, and/or couple to the prosthetic valve 1005.

In some examples, the petals 1025 may be formed by bending and/or otherwise altering a shape and/or direction of one or more struts forming the frame 1002. The frame 1002 may be at least partially flared to increase an extension of the one or more petals 1025.

The distal end 1018 may advantageously be configured to form a seal with a junction area between a blood vessel (e.g., the IVC) and a chamber (e.g., the right atrium). Sealing against the anatomy can help prevent reverse caval blood flow that can cause peripheral and/or venous congestion.

In some examples, the implant 1000 may comprise one or more anchoring elements (e.g., hooks, pegs, arms, fingers, etc.) extending outwardly from the frame 1002 and/or configured to improve anchoring of the implant 1000 with the IVC geometry to prevent migration of the implant 1000 upward (e.g., into the right atrium) and/or downward (e.g., into the IVC).

The implant 1000 may be configured to be deployed in any suitable manner. For example, the implant 1000 may be deployed in two stages. First, the frame 1002 and/or sealing element 1004 may be deployed and/or anchored at least partially in the blood vessel. Once the frame 1002 and/or sealing element 1004 is in place, the prosthetic valve 1005 may be docked to the frame 1002 and/or sealing element 1004. Alternatively, the valve 1005 may be coupled to the frame 1002 and/or sealing element 1004 prior to delivery into the body. In some cases, coupling the valve 1005 to the frame 1002 and/or sealing element 1004 may allow for consolidating a procedure and/or reducing delivery steps of the procedure.

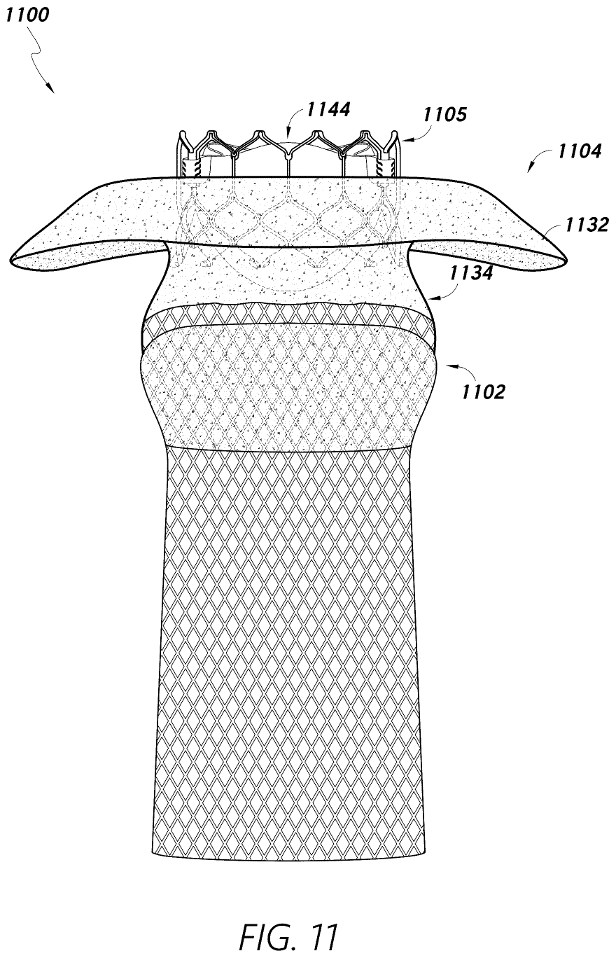

FIG. 11 illustrates another example implant 1100 configured for anchoring into one or more blood vessels and/or docking one or more prosthetic valves 1105 in accordance with one or more examples. The implant 1100 may comprise a frame 1102 (e.g., comprising a network of wires and/or struts forming one or more cells) and/or a sealing element 1004 disposed at a distal end of the frame 1102 and/or implant 1100.

In some examples, the sealing element 1104 may be at least partially covered. For example, the frame 1102 may comprise a wireframe at least partially enclosed by a covering comprising one or more fabrics and/or other materials. In some examples, the sealing element 1104 may be configured to have a greater diameter than the frame 1102. For example, the sealing element 1104 may be configured to extend approximately 5-10 mm wider than the frame 1102.

The sealing element 1104 may comprise a proximal portion 1134 and/or a distal portion 1132. The distal portion 1132 may be configured to form an umbrella-like covering with a greater diameter than the proximal portion 1134. In some examples, the distal portion 1132 may be configured to drape and/or curve back over the proximal portion 1134 and/or to at least partially enclose the proximal portion 1134. The proximal portion 1134 may extend approximately 3-5 mm between the distal portion 1132 and the frame 1102. The frame 1102 may be coupled to the proximal portion 1134 and/or may have a length of approximately 8-10 cm.

In some examples, the frame 1102 may form a bulb and/or bulge along a length of the frame 1102 to improve anchoring and/or sealing of the frame 1102. For example, the frame 1102 may comprise a bulb and/or bulge beginning approximately 10-40 mm below the sealing element 1104 and/or extending for approximately 10-20 mm along a length of the frame 1102. In some examples, an example bulb and/or bulge may have an increased diameter and/or may extend radially approximately 5-20 mm beyond a width of other portions of the frame 1102.

The frame 1102 and/or sealing element 1104 may be coupled to the valve 1105 and/or the valve 1105 may be configured for docking to the frame 1102 and/or sealing element 1104 following delivery of the frame 1102 and/or sealing element 1104. The valve 1105 may comprise one or more leaflets 1144.

The implant 1100 may be configured for antegrade delivery (e.g., via the femoral vein) and/or retrograde delivery (e.g., via the subclavian and/or jugular vein). The distal portion 1132 of the sealing element 1104 may be extruded out of the frame 1102 following delivery of the frame 1102 and sealing element 1104 and/or may be configured to form a seal at a junction (e.g., the right atrium IVC junction) as the implant 1100 is pulled (e.g., for antegrade delivery) and/or pushed (e.g., for retrograde delivery) into place such that the distal portion 1132 of the sealing element 1104 rests along a floor of the right atrium and/or other chamber. The distal portion 1132 of the sealing element 1104 may provide an extension of a flared distal end of the frame 1102. In some examples, a width and/or length of the distal portion 1132 of the sealing element 1104 may be sufficiently small to avoid interaction and/or blockage of the valve 1105 and/or other blood vessels (e.g., the coronary sinus).

After the distal portion 1132 of the sealing element 1104 is extruded and/or expanded, the frame 1102 may similarly be expanded in width and/or length to fill the blood vessel. A length of the proximal portion 1134 of the sealing element 1104 may be configured to allow the valve 1105 to seal within the implant 1100.

In some examples, the frame 1102 may comprise various anchoring features. For example, the frame 1102 may comprise anchoring arms, fingers, tines, hooks, and/or other elements configured to anchor the frame 1102 to blood vessel walls. The frame 1102 may comprise a bulb and/or bulge extending outwardly from the frame 1102 (e.g., at a sub-hepatic surface of the frame 1102) to improve security and/or fit with the anatomy.

The implant 1100 may comprise one or more markers to facilitate delivery of the implant 1100. For example, the implant 1100 may comprise one or more tantalum markers below the distal portion 1132 of the sealing element 1104 to facilitate placement of the distal portion 1132 of the sealing element 1104 along a floor of the right atrium and/or other chamber and/or to facilitate positioning above a highest hepatic vein and/or other branching blood vessel.

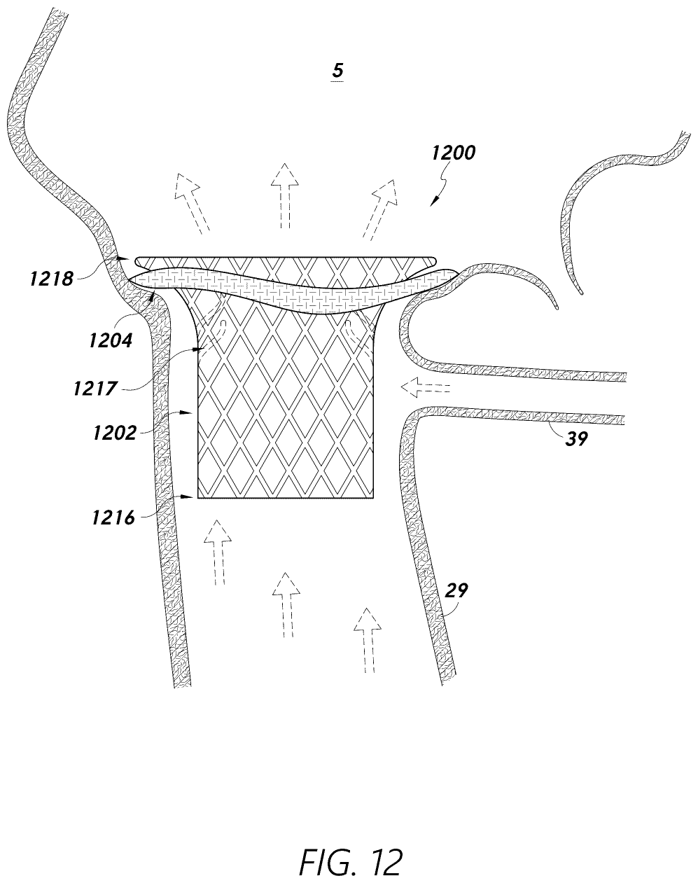

FIG. 12 illustrates an example implant 1200 anchored into one or more blood vessels 29 (e.g., the IVC) and/or configured to dock one or more prosthetic valves in accordance with one or more examples. The implant 1200 may comprise a frame 1202 (e.g., comprising a network of wires and/or struts forming one or more cells) and/or a sealing element 1004 disposed at a distal end of the frame 1202 and/or implant 1200.

In some examples, the sealing element 1204 may be at least partially covered. For example, the sealing element 1204 may comprise a wire frame at least partially enclosed by a covering comprising one or more fabrics and/or other materials. In some examples, the sealing element 1204 may be configured to have a greater diameter than the frame 1202. For example, the sealing element 1204 may be configured to extend approximately 5-10 mm wider than the frame 1202.

The sealing element 1204 may comprise a proximal portion and/or a distal portion. The distal portion may be configured to form an umbrella-like covering with a greater diameter than the proximal portion. In some examples, the distal portion may be configured to drape and/or curve back over the proximal portion and/or to at least partially enclose the proximal portion. The proximal portion may extend approximately 3-5 mm between the distal portion and the frame 1202. The frame 1202 may be coupled to the proximal portion and/or may have a length of approximately 8-10 cm.

In some examples, the frame 1202 may form a bulb and/or bulge along a length of the frame 1202 to improve anchoring and/or sealing of the frame 1202. For example, the frame 1202 may comprise a bulb and/or bulge beginning approximately 10-40 mm below the sealing element 1204 and/or extending for approximately 10-20 mm along a length of the frame 1202. In some examples, an example bulb and/or bulge may have an increased diameter and/or may extend radially approximately 5-20 mm beyond a width of other portions of the frame 1202.

The frame 1202 and/or sealing element 1204 may be coupled to the valve and/or the valve may be configured for docking to the frame 1202 and/or sealing element 1204 following delivery of the frame 1202 and/or sealing element 1204. The valve may comprise one or more leaflets.

The implant 1200 may be configured for antegrade delivery (e.g., via the femoral vein) and/or retrograde delivery (e.g., via the subclavian and/or jugular vein). The distal portion of the sealing element 1204 may be extruded out of the frame 1202 following delivery of the frame 1202 and sealing element 1204 and/or may be configured to form a seal at a junction (e.g., the right atrium 5 IVC 29 junction) as the implant 1200 is pulled (e.g., for antegrade delivery) and/or pushed (e.g., for retrograde delivery) into place such that the distal portion of the sealing element 1204 rests along a floor of the right atrium 5 and/or other chamber. The distal portion of the sealing element 1204 may provide an extension of a flared distal end of the frame 1202. In some examples, a width and/or length of the distal portion of the sealing element 1204 may be sufficiently small to avoid interaction and/or blockage of the valve and/or other blood vessels (e.g., the coronary sinus).

After the distal portion of the sealing element 1204 is extruded and/or expanded, the frame 1202 may similarly be expanded in width and/or length to fill the blood vessel 29. A length of the proximal portion of the sealing element 1204 may be configured to allow the valve to seal within the implant 1200.

In some examples, the frame 1202 may comprise various anchoring features. For example, the frame 1202 may comprise anchoring arms, fingers, tines, hooks, and/or other elements configured to anchor the frame 1202 to blood vessel walls. The frame 1202 may comprise a bulb and/or bulge extending outwardly from the frame 1202 (e.g., at a sub-hepatic surface of the frame 1202) to improve security and/or fit with the anatomy.

The implant 1200 may comprise one or more markers to facilitate delivery of the implant 1200. For example, the implant 1200 may comprise one or more tantalum markers below the distal portion of the sealing element 1204 to facilitate placement of the distal portion of the sealing element 1204 along a floor of the right atrium 5 and/or other chamber and/or to facilitate positioning above a highest hepatic vein 39 and/or other branching blood vessel.

The sealing element 1204 may be configured to be disposed below the distal end 1218 of the frame 1202 and/or below a flared portion of the distal end 1218. The sealing element 1204 may be configured to provide a flange, lip, and/or seal at or near the distal end 1218. In some examples, the sealing element 1204 may comprise similar materials and/or a similar structure as the frame 1202. For example, the sealing element 1204 and/or frame 1202 may comprise Nitinol and/or other materials and/or may be at least partially covered in Dacron and/or other materials. A proximal end 1216 of the frame 1202 may be flared and/or may have generally straight sides and/or consistent diameter relative to a midsection of the frame 1202.

The sealing element 1204 may comprise a compressible and/or conformable flange and/or anchor at a top and/or distal end 1218 of the frame 1202. The sealing element 1204 may be configured to compress while within a catheter and/or other delivery device and/or may be configured to expand upon removal and/or in response to the frame 1202 being pulled down into position.

In some examples, the implant 1200 may comprise one or more inward-extending arms 1217 configured to couple to and/or grasp one or more prosthetic valves. For example, a prosthetic valve may be configured to dock within a lumen of the frame 1202 and/or the arms 1217 may be configured to extend into the lumen to contact the valve.

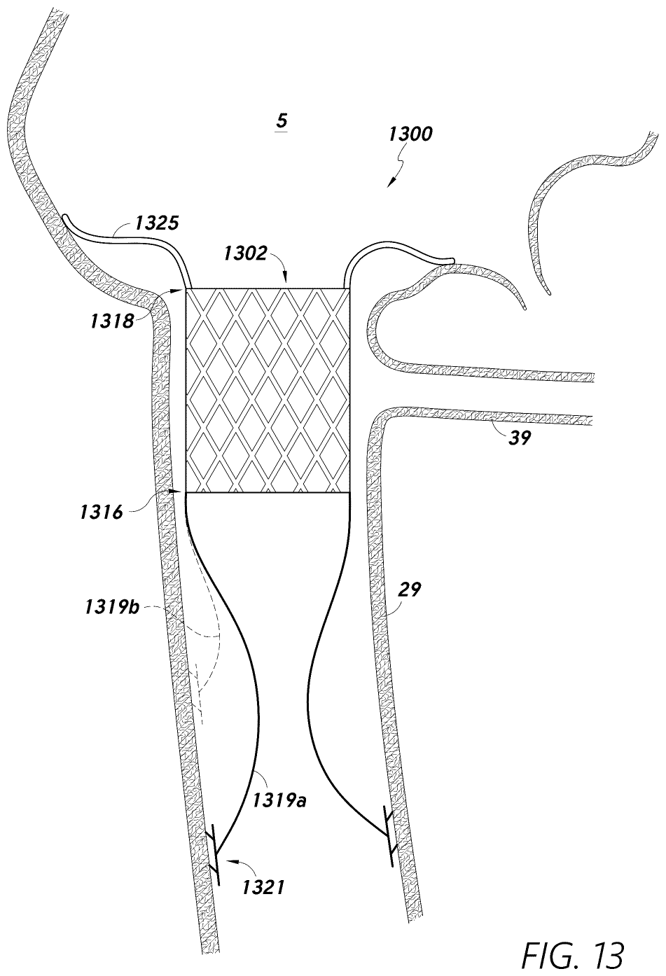

FIG. 13 illustrates another example implant 1300 configured for placement at least partially within a blood vessel 29 (e.g., the IVC) and/or configured to dock one or more prosthetic valves in accordance with one or more examples herein. The implant 1300 may comprise a frame 1302 (e.g., a wire frame) comprising a network of struts forming one or more cells to allow lateral blood flow from one or more branching blood vessels 39 into the implant 1300. The implant 1300 may comprise one or distal arms 1325 configured to extend laterally and/or distally from the distal end 1318 of the frame and/or along a floor of the right atrium 5 and/or other chamber. In some examples, the one or more arms 1325 may be covered in a cloth and/or other material to improve friction of the one or more arms 1325.

The implant 1300 may additionally or alternatively comprise one or more proximal arms 1319 configured to extend from a proximal end 1316 the of the frame 1302. The one or more proximal arms 1319 may comprise anchors 1321, which can include barbed ends. In some examples, different proximal arms 1319 may have different lengths, shapes, and/or orientations. For example, the implant 1300 may comprise a first proximal arm 1319a having a greater length than a second proximal arm 1319b and/or the first proximal arm 1319a may be configured to extend deeper into the blood vessel 29 than the second proximal arm 1319b.

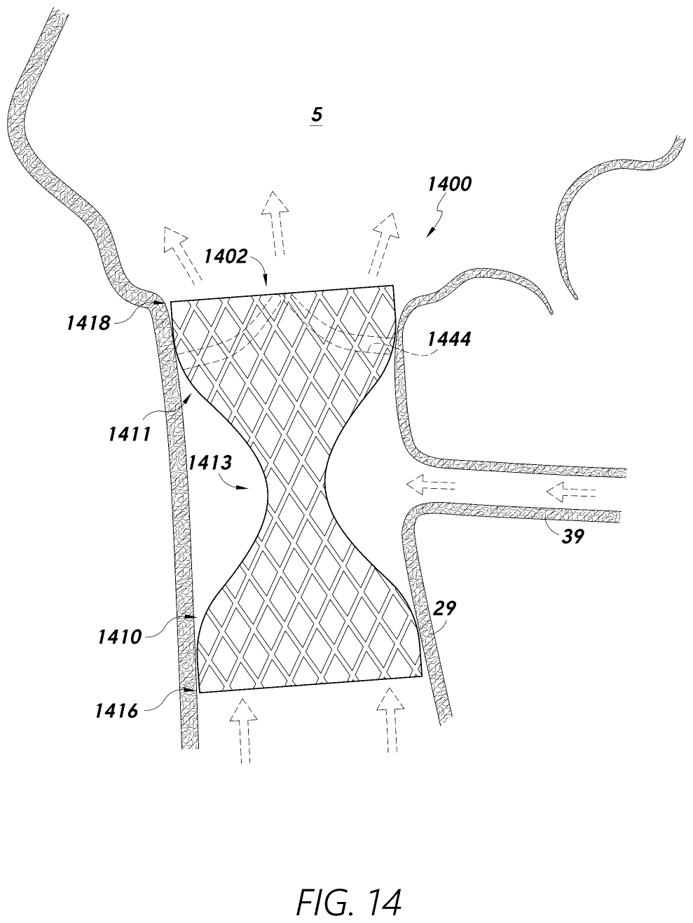

FIG. 14 illustrates another example implant 1400 disposed within a blood vessel 29 in accordance with one or more examples. The implant 1400 may comprise a frame 1402 having a midsection 1413 comprising inwardly bent walls forming a reduced diameter and/or width relative to a proximal end 1416 and/or distal end 1418 of the implant 1400. The midsection 1413 may be configured to be disposed at or near inflow junctions of one or more branching blood vessels 39. In some examples, the inwardly bent walls of the midsection 1413 may be configured to minimize interaction between the frame 1402 and the one or more branching blood vessels 39. The midsection 1413 may have a reduced diameter relative to a proximal portion 1410 and/or a distal portion 1411 of the frame 1402.

The implant 1400 may be configured to receive one or more prosthetic valves and/or may comprise one or more prosthetic valves and/or valve components. For example, the implant 1400 may comprise one or more leaflets 1444 (e.g., two or three) extending across a lumen of the frame 1402. The inclusion of leaflets 1444 with the frame 1402 may advantageously limit delivery steps and/or processes. The leaflets 1444 may be coupled to the frame 1402 and/or extend from the frame 1402.

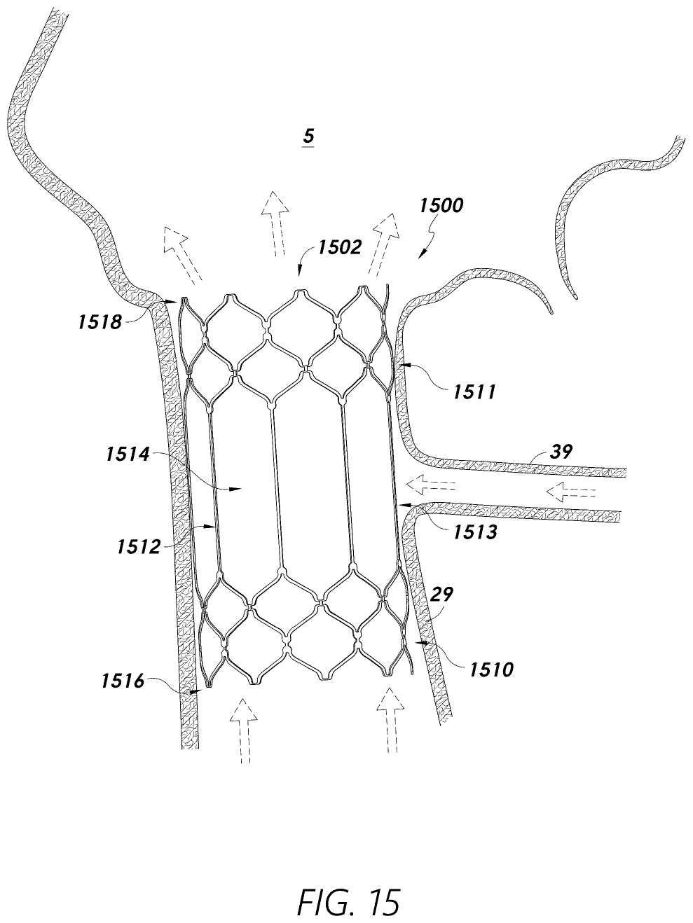

FIG. 15 illustrates another example implant 1500 disposed within a blood vessel 29 in accordance with one or more examples. In some examples, the implant 1500 may comprise a frame 1502 comprising a network of struts 1512 forming one or more cells 1514 and/or openings through and/or into the frame 1502. The frame 1502 may comprise variably sized cells 1514. For example, the struts 1512 may form generally larger and/or longer cells 1514 at or near a midsection of the frame 1502. The larger cells 1514 may be configured for placement adjacent to one or more branching blood vessels 39 to reduce contact with and/or obstruction of the branching blood vessels 39. One or more cells 1514 at or near a distal end 1518 and/or proximal end 1516 of the frame 1502 may be smaller than the cells 1514 at the midsection.

The larger cells 1514 may be disposed at a midsection 1513 of the frame 1502. The midsection 1513 may be disposed between a proximal section 1510 and/or a distal section 1511 of the frame 1502. The proximal section 1510 and/or distal section 1511 may comprise one or more cells 1514 having smaller sizes relative to the cells 1514 at the midsection 1513.

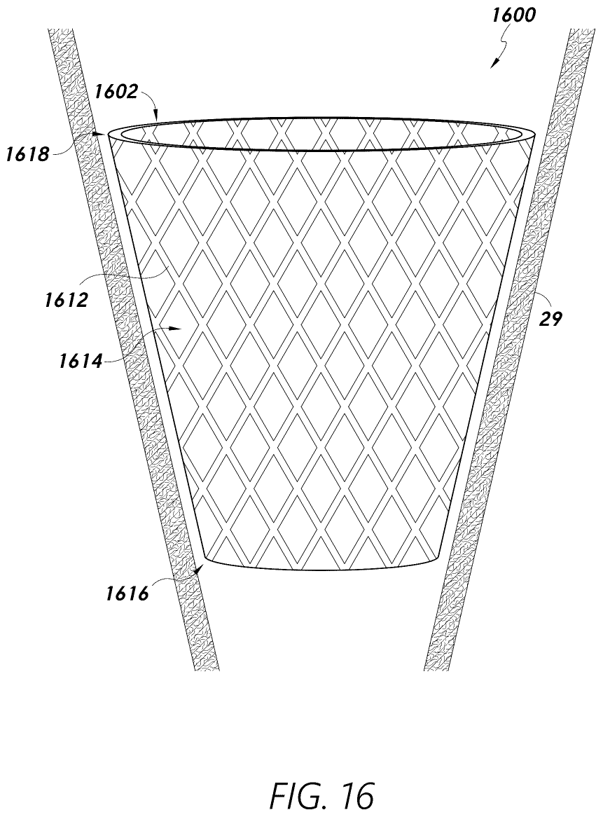

FIG. 16 illustrates another example implant 1600 disposed within a blood vessel 29 in accordance with one or more examples. The implant 1600 may comprise a frame 1602 having a generally tapered form. For example, a diameter of the frame 1602 may gradually reduce from a distal end 1618 of the frame 1602 to a proximal end 1616 of the frame 1602. The implant 1600 may be configured to track and/or approximate a tapered shape of the blood vessel 29 (e.g., the IVC). For example, the blood vessel 29 may increase in diameter towards a junction of the blood vessel 29 and another blood vessel and/or chamber.

In some examples, the implant 1600 may comprise one or more sealing elements. The shape of the implant 1600 may allow for components of the frame 1602 to more closely interact and/or anchor to the blood vessel 29.

The implant 1600 may comprise a network of struts 1612 forming one or more cells 1614. The cells 1614 may have generally uniform sizes and/or shapes and/or may be diamond shaped.

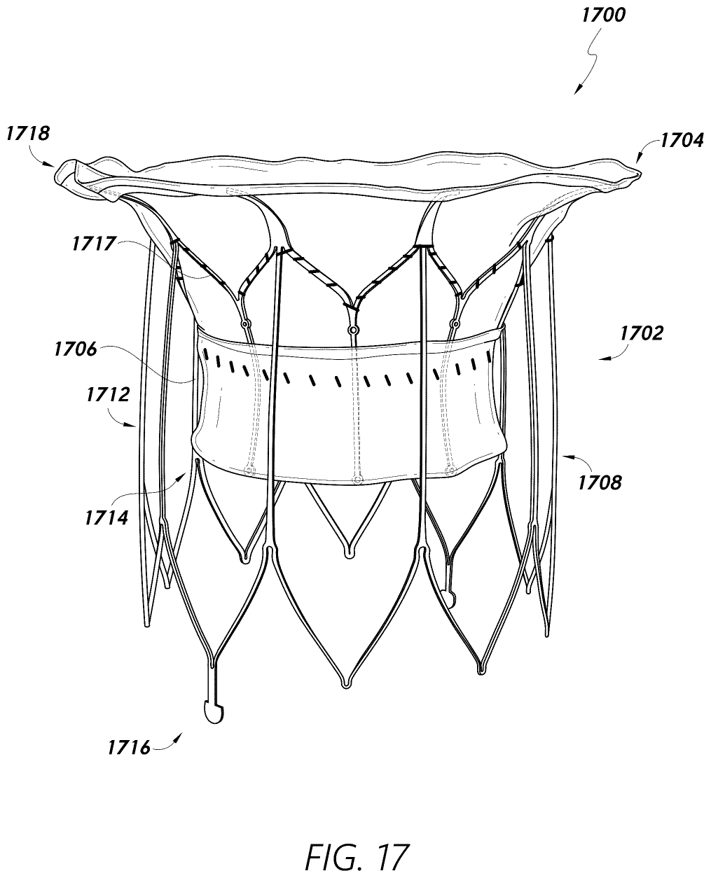

FIG. 17 illustrates components of another example implant 1700 configured to dock and/or support one or more prosthetic valves and/or valve components in accordance with one or more instances. The implant 1700 may comprise a frame 1702 and/or a sealing element 1704, which can include a skirt, covering, and/or similar device. The frame 1702 may be configured to form an inner frame 1706 and/or an outer frame 1708. The inner frame 1706 may form a first diameter that is less than a second diameter of the outer frame 1708. The inner frame 1706 and outer frame 1708 may be extensions of a common device and/or may extend from each other.

The frame 1702 may comprise a wire frame formed by a network of struts 1712 (e.g., wires, cords, and/or bars) forming one or more cells 1714. The frame 1702 may mostly comprise empty space and/or cells 1714 between the struts 1712. For example, the struts 1712 may be generally thin and/or may be spaced apart to create relatively large gaps between the struts 1712, as shown in FIG. 17. In some examples, the outer frame 1708 may comprise a series of longitudinally extending struts 1712 aligned in series and/or in parallel around a circumference of the implant 1700. Each of the struts 1712 may join to one or more adjacent struts 1712 at a proximal end 1716 of the implant 1700 and/or may join with one or more adjacent struts 1712 and/or with the inner frame 1706 at a distal end 1718 of the implant 1700.

The inner frame 1706 may similarly comprise a network of generally thin, elongate, and/or spaced apart struts 1712. In some examples, one or more struts 1712 of the inner frame 1706 may extend generally in series and/or in parallel longitudinally along a length of the implant 1700. The one or more struts 1712 of the inner frame 1706 may be disposed between struts 1712 of the outer frame 1708.

In some examples, the outer frame 1708 may have a generally cylindrical form and/or may at least partially enclose a complete circumference of the inner frame 1706 and/or of a portion of the sealing element 1704. The outer frame 1708 may comprise a network of struts 1712, which can include wires, arms, bars, cords, walls, and/or similar components forming one or more cells 1714 and/or openings through the outer frame 1708. The one or more cells 1714 may be configured to allow blood flow through the outer frame 1708. The cells 1714 may have any suitable shape and/or size. The outer frame 1708 may form generally elongate cells 1714 extending approximately an entire length of the outer frame 1708 and/or from a proximal end 1716 to a distal end 1718 of the implant 1700. The one or more cells 1714 may have triangular forms at end points of the one or more cells 1714. However, the one or more struts 1712 may form cells 1714 having different shapes. For example, the struts 1712 may be configured to form generally rectangular and/or diamond-shaped cells 1714. While the struts 1712 are shown having generally thick structures, the one or more struts 1712 may have wire-like and/or generally thin forms. In some examples, the inner frame 1706 and/or outer frame 1708 may be configured to maintain a uniform structure and/or strut 1712 pattern along a length of the frame 1702.

The implant 1700 may be configured for delivery and/or placement at an SVC and/or IVC of a heart. For example, the implant 1700 may be configured for placement at or near an in-flow junction of the SVC and/or IVC to the right atrium of the heart.

At a distal end 1718 of the implant 1700, the outer frame 1708 and inner frame 1706 may join together and/or the inner frame 1706 may extend away from the outer frame 1708 and/or along an inner lumen formed by the outer frame 1708. In some examples, the outer frame 1708 and inner frame 1706 may both have a generally flared and/or conical form at or near the distal end 1718 of the implant 1700. The inner frame 1706 may have a flaring angle that is greater than the outer frame 1708 such that the diameter of the inner frame 1706 may be less than the diameter of the outer frame 1708. The inner frame 1706 may be configured to extend along at least a portion of the length of the outer frame 1708.

The flared end (e.g., distal end 1718) of the outer frame 1708 and/or inner frame 1706 may be configured to engage an atrium and/or other chamber when implanted. The sealing element 1704 may be configured to extend along the inner frame 1706 and/or may be configured to wrap around the outer frame 1708 and/or extend between the outer frame 1708 and the native tissue. The outer frame 1708 may be configured to extend into the IVC and/or other blood vessel and/or the sealing element 1704 may be configured to extend from the outer frame 1708 to an inner surface of the inner frame 1706. The sealing element 1704 may be configured for engagement with a prosthetic valve and/or other implant. For example, the sealing element 1704 may be configured to provide a mounting surface for a prosthetic valve and/or may be configured to securely hold the prosthetic valve. The sealing element 1704 may be configured to extend along only a portion of the implant 1700 such that the sealing element 1704 may not extend across one or more branching vessels of the blood vessel.

In some examples, the implant 1700 may comprise one or more outward bulbs configured to extend outwardly from the diameter of the outer frame 1708 to facilitate anchoring of the implant 1700 within a blood vessel.

The frame 1702 may comprise one or more downward-extending arms 1717 (e.g., extending towards the proximal end 1716) configured to form the inner frame 1706. For example, the arms 1717 may extend downwardly from the outer frame 1708 at or near the distal end 1718 of the frame 1702. The one or more arms 1717 may be configured to extend at an acute angle away from the outer frame into the lumen of the frame 1702 and/or may extend generally in parallel with the outer frame 1708 at or near distal ends of the one or more arms 1717.

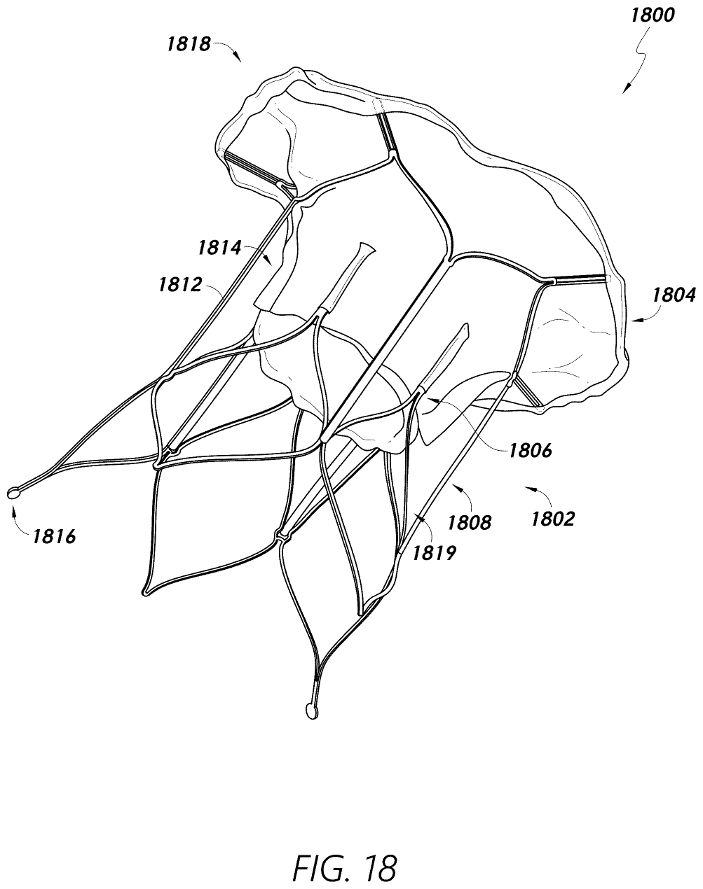

FIG. 18 illustrates components of another example implant 1800 configured to dock and/or support one or more prosthetic valves and/or valve components in accordance with one or more instances. The implant 1800 may comprise a frame 1802 and/or a sealing element 1804, which can include a skirt, covering, and/or similar device. The frame 1802 may be configured to form an inner frame 1806 and/or an outer frame 1808. The inner frame 1806 may form a first diameter that is less than a second diameter of the outer frame 1808. The inner frame 1806 and outer frame 1808 may be extensions of a common device and/or may extend from each other.

The frame 1802 may comprise a wire frame formed by a network of struts 1812 (e.g., wires, cords, and/or bars) forming one or more cells 1814. The frame 1802 may mostly comprise empty space and/or cells 1814 between the struts 1812. For example, the struts 1812 may be generally thin and/or may be spaced apart to create relatively large gaps between the struts 1812, as shown in FIG. 18. In some examples, the outer frame 1808 may comprise a series of longitudinally extending struts 1812 aligned in series and/or in parallel around a circumference of the implant 1800. Each of the struts 1812 may join to one or more adjacent struts 1812 at a proximal end 1816 of the implant 1800 and/or may join with one or more adjacent struts 1812 and/or with the inner frame 1806 at a distal end 1818 of the implant 1800.

The inner frame 1806 may similarly comprise a network of generally thin, elongate, and/or spaced apart struts 1812. In some examples, one or more struts 1812 of the inner frame 1806 may extend generally in series and/or in parallel longitudinally along a length of the implant 1800. The one or more struts 1812 of the inner frame 1806 may be disposed between struts 1812 of the outer frame 1808.

In some examples, the outer frame 1808 may have a generally cylindrical form and/or may at least partially enclose a complete circumference of the inner frame 1806 and/or of a portion of the sealing element 1804. The outer frame 1808 may comprise a network of struts 1812, which can include wires, arms, bars, cords, walls, and/or similar components forming one or more cells 1814 and/or openings through the outer frame 1808. The one or more cells 1814 may be configured to allow blood flow through the outer frame 1808. The cells 1814 may have any suitable shape and/or size. The outer frame 1808 may form generally elongate cells 1814 extending approximately an entire length of the outer frame 1808 and/or from a proximal end 1816 to a distal end 1818 of the implant 1800. The one or more cells 1814 may have triangular forms at end points of the one or more cells 1814. However, the one or more struts 1812 may form cells 1814 having different shapes. For example, the struts 1812 may be configured to form generally rectangular and/or diamond-shaped cells 1814. While the struts 1812 are shown having generally thick structures, the one or more struts 1812 may have wire-like and/or generally thin forms. In some examples, the inner frame 1806 and/or outer frame 1808 may be configured to maintain a uniform structure and/or strut 1812 pattern along a length of the frame 1802.

The implant 1800 may be configured for delivery and/or placement at an SVC and/or IVC of a heart. For example, the implant 1800 may be configured for placement at or near an in-flow junction of the SVC and/or IVC to the right atrium of the heart.

At a distal end 1818 of the implant 1800, the outer frame 1808 and inner frame 1806 may join together and/or the inner frame 1806 may extend away from the outer frame 1808 and/or along an inner lumen formed by the outer frame 1808. In some examples, the outer frame 1808 and inner frame 1806 may both have a generally flared and/or conical form at or near the distal end 1818 of the implant 1800. The inner frame 1806 may have a flaring angle that is greater than the outer frame 1808 such that the diameter of the inner frame 1806 may be less than the diameter of the outer frame 1808. The inner frame 1806 may be configured to extend along at least a portion of the length of the outer frame 1808.

The flared end (e.g., distal end 1818) of the outer frame 1808 and/or inner frame 1806 may be configured to engage an atrium and/or other chamber when implanted. The sealing element 1804 may be configured to extend along the inner frame 1806 and/or may be configured to wrap around the outer frame 1808 and/or extend between the outer frame 1808 and the native tissue. The outer frame 1808 may be configured to extend into the IVC and/or other blood vessel and/or the sealing element 1804 may be configured to extend from the outer frame 1808 to an inner surface of the inner frame 1806. The sealing element 1804 may be configured for engagement with a prosthetic valve and/or other implant. For example, the sealing element 1804 may be configured to provide a mounting surface for a prosthetic valve and/or may be configured to securely hold the prosthetic valve. The sealing element 1804 may be configured to extend along only a portion of the implant 1800 such that the sealing element 1804 may not extend across one or more branching vessels of the blood vessel.

In some examples, the implant 1800 may comprise one or more outward bulbs configured to extend outwardly from the diameter of the outer frame 1808 to facilitate anchoring of the implant 1800 within a blood vessel.

The frame 1802 may comprise one or more upward-extending arms 1819 (e.g., extending towards the distal end 1818) configured to form the inner frame 1806. For example, the arms 1819 may extend downwardly from the outer frame 1808 at or near the distal end 1818 of the frame 1802. The one or more arms 1819 may be configured to extend at an acute angle away from the outer frame into the lumen of the frame 1802 and/or may extend generally in parallel with the outer frame 1808 at or near distal ends of the one or more arms 1819.

The arms 1819 of the implant 1800 may extend upwardly from the proximal end 1816 to the distal end 1818. For example, the one or more arms 1819 may extend at an approximately 45-degree angle from the outer frame 1808 at or near the distal end 1818 of the implant 1800 and/or may generally upwardly generally in parallel with the outer frame 1808 along a midsection of the implant 1800.

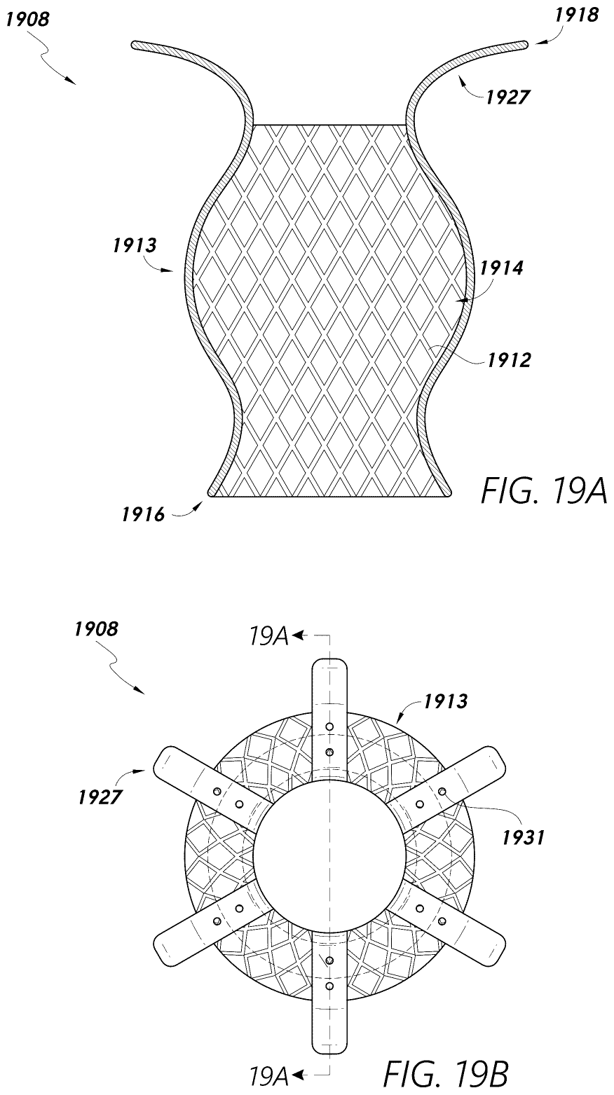

FIGS. 19A and 19B illustrate an outer frame 1908 of an example implant configured to dock and/or support one or more prosthetic valves and/or valve components in accordance with one or more instances. FIG. 19A provides a cross-sectional side view of the outer frame 1908 and FIG. 19B provides an overhead view of the outer frame 1908. The outer frame 1908 may comprise one or more flared and/or elongate arms 1927 at a distal end 1918 of the outer frame 1908 and/or configured to extend outwardly and/or distally with respect to the outer frame 1908. In the example shown in FIG. 19B, the outer frame 1908 comprises six arms 1927. However, the outer frame 1908 may comprise any number of arms 1927. In some examples, the arms 1927 may be configured to receive and/or to provide a platform and/or docking support for one or more sealing elements.

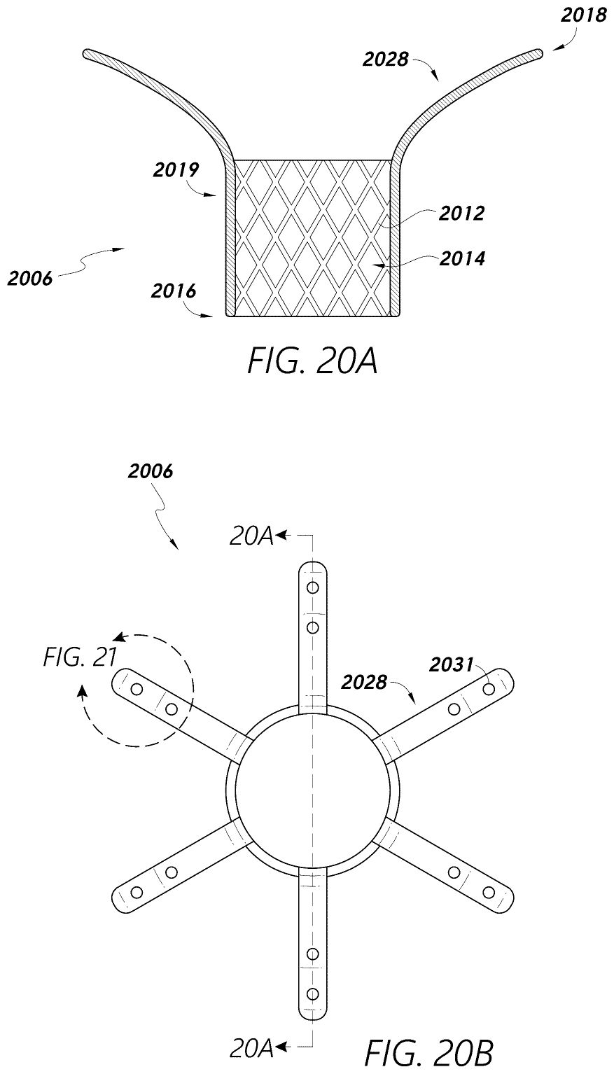

In some examples, the outer frame 1908 may be configured to attach to and/or mate with a corresponding inner frame (see, e.g., FIGS. 20A and 20B). For example, the arms 1927 of the outer frame 1908 may be configured to attach to corresponding arms of the inner frame. In some examples, the outer frame 1908 and/or inner frame may comprise various features configured to facilitate attachment and/or mating between the outer frame 1908 and the inner frame. For example, the outer frame 1908 and/or inner frame may comprise one or more apertures 1931 and/or cavities configured to receive one or more binding and/or attachment tools and/or devices, which can include wires, strings, sutures, cords, and/or similar devices. While the outer frame 1908 is shown comprising two apertures 1931 aligned longitudinally along each arm 1927, the outer frame 1908 and/or arms 1927 of the outer frame 1908 may comprise any number and/or orientation of apertures 1931.

In some examples, the outer frame 1908 may have a generally cylindrical form and/or may at least partially enclose a complete circumference of an inner frame and/or of a portion of a sealing element attached to the outer frame 1908. The outer frame 1908 may comprise a network of struts 1912, which can include wires, arms, bars, cords, walls, and/or similar components forming one or more cells 1914 and/or openings through the outer frame 1908. The one or more cells 1914 may be configured to allow blood flow through the outer frame 1908. The cells 1914 may have any suitable shape and/or size. The outer frame 1908 may form generally elongate cells 1914 extending approximately an entire length of the outer frame 1908 and/or from a proximal end 1916 to a distal end 1918 of the implant 1900. The one or more cells 1914 may be diamond shaped and/or may have any suitable shape. While the struts 1912 are shown having generally thick structures, the one or more struts 1912 may have wire-like and/or generally thin forms.

The outer frame 1908 may be configured for delivery and/or placement at an SVC and/or IVC of a heart. For example, the outer frame 1908 may be configured for placement at or near an in-flow junction of the SVC and/or IVC to the right atrium of the heart.

The outer frame 1908 may be configured to prevent blockage of one or more branching blood vessels (e.g., hepatic veins) when implanted within a main blood vessel (e.g., the IVC). For example, the outer frame 1908 may be configured to allow blood flow through the outer frame 1908 between the proximal end 1916 and the distal end 1918.

The outer frame 1908 may comprise one or more flared ends and/or arms 1927 at the distal end 1918 and/or proximal end 1916. In some examples, the outer frame 1908 may be laser-cut and/or may comprise one or more shape memory alloys (e.g., Nitinol).

The outer frame 1908 may have any suitable shape. In some examples, the outer frame 1908 may have an hourglass shape and/or may comprise outward extending sides forming a bulge 1913 at or near a midpoint of the outer frame 1908. The proximal end 1916 and/or distal end 1918 may be generally flared and/or the outer frame 1908 may have generally inwardly extending sides between the bulge 1913 and the distal end 1918 and/or between the bulge 1913 and the proximal end 1916.

FIGS. 20A and 20B illustrate an inner frame 2006 of an example implant configured to dock and/or support one or more prosthetic valves and/or valve components in accordance with one or more instances. FIG. 20A provides a cross-sectional side view of the inner frame 2006 and FIG. 20B provides an overhead view of the inner frame 2006. The inner frame 2006 may comprise one or more flared and/or elongate arms 2028 at a distal end 2018 of the inner frame 2006 and/or configured to extend outwardly and/or distally with respect to the inner frame 2006. In the example shown in FIG. 20B, the inner frame 2006 comprises six arms 2028. However, the inner frame 2006 may comprise any number of arms 2028. In some examples, the arms 2028 may be configured to receive and/or to provide a platform and/or docking support for one or more sealing elements.

In some examples, the inner frame 2006 may be configured to attach to and/or mate with a corresponding outer frame (see, e.g., FIGS. 19A and 19B). For example, the arms 2028 of the inner frame 2006 may be configured to attach to corresponding arms of the outer frame. In some examples, the outer frame and/or inner frame 2006 may comprise various features configured to facilitate attachment and/or mating between the outer frame and the inner frame 2006. For example, the outer frame and/or inner frame 2006 may comprise one or more apertures 2031 and/or cavities configured to receive one or more binding and/or attachment tools and/or devices, which can include wires, strings, sutures, cords, and/or similar devices. While the inner frame 2006 is shown comprising two apertures 2031 aligned longitudinally along each arm 2028, the inner frame 2006 and/or arms 2028 of the inner frame 2006 may comprise any number and/or orientation of apertures 2031.

In some examples, the inner frame 2006 may have a generally cylindrical form and/or may be configured to fit at least partially within an outer frame. The inner frame 2006 may comprise a generally cylindrical and/or tubular body 2019 portion comprising a network of struts 2012, which can include wires, arms, bars, cords, walls, and/or similar components forming one or more cells 2014 and/or openings through the inner frame 2006. The one or more cells 2014 may be configured to allow blood flow through the inner frame 2006. The cells 2014 may have any suitable shape and/or size. The inner frame 2006 may form generally elongate cells 2014 extending approximately an entire length of the inner frame 2006 and/or from a proximal end 2016 to a distal end 2018 of the implant 2000. The one or more cells 2014 may be diamond shaped and/or may have any suitable shape. While the struts 2012 are shown having generally thick structures, the one or more struts 2012 may have wire-like and/or generally thin forms.

The inner frame 2006 may be configured for delivery and/or placement at an SVC and/or IVC of a heart. For example, the inner frame 2006 may be configured for placement at or near an in-flow junction of the SVC and/or IVC to the right atrium of the heart.

The inner frame 2006 may be configured to prevent blockage of one or more branching blood vessels (e.g., hepatic veins) when implanted within a main blood vessel (e.g., the IVC). For example, the inner frame 2006 may be configured to allow blood flow through the inner frame 2006 between the proximal end 2016 and the distal end 2018. When coupled to an outer frame, the inner frame 2006 and/or the body 2019 may be configured to extend at least partially away from the outer frame and/or to form a gap between the struts of the outer frame and the struts 2012 of the inner frame 2006.

The inner frame 2006 may comprise one or more flared ends and/or arms 2028 at the distal end 2018 and/or proximal end 2016. In some examples, the inner frame 2006 may be laser-cut and/or may comprise one or more shape memory alloys (e.g., Nitinol).

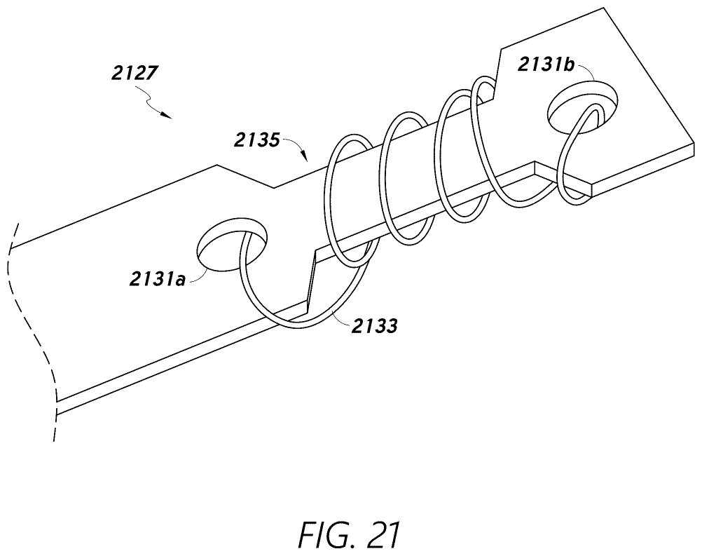

FIG. 21 illustrates at least a portion of an example arm 2127 of an implant (e.g., an outer frame and/or inner frame of the implant), in accordance with one or more examples herein. The arm 2127 may have a generally flared and/or elongate form and/or may be configured to extend from a distal end of an inner frame and outer frame. Moreover, the arm 2127 may be configured to extend outwardly and/or distally with respect to the inner frame and/or outer frame. In some examples, the arm 2127 may be configured to receive and/or to provide a platform and/or docking support for one or more sealing elements.

In some examples, the arm 2127 may be configured to attach to other arms of the implant. In some examples, the implant may comprise an outer frame and/or inner frame. The outer frame may be at least partially disconnected from the inner frame. In some examples, the arm 2127 and/or the outer frame and/or inner frame may comprise various features configured to facilitate attachment and/or mating between the outer frame and the inner frame. For example, the arm 2127 may comprise one or more apertures 2131 and/or cavities configured to receive one or more cords 2133, which can include wires, strings, sutures, and/or similar devices. While the arm 2127 is shown comprising two apertures 2031 aligned longitudinally along each the arm 2127, the arm 2127 may comprise any number and/or orientation of apertures 2031.

The arm 2127 may have a variable width and/or may comprise multiple sections. For example, the arm 2127 may comprise a neck 2135 disposed between a first aperture 2131a and a second aperture 2131b of the arm 2127. The neck 2135 may have a reduced width relative to other portions of the arm 2127. The neck 2135 may be configured to support one or more winds of the cord 2133. Arms 2127 of an outer frame and/or inner frame may comprise a neck 2135 having a reduced width.

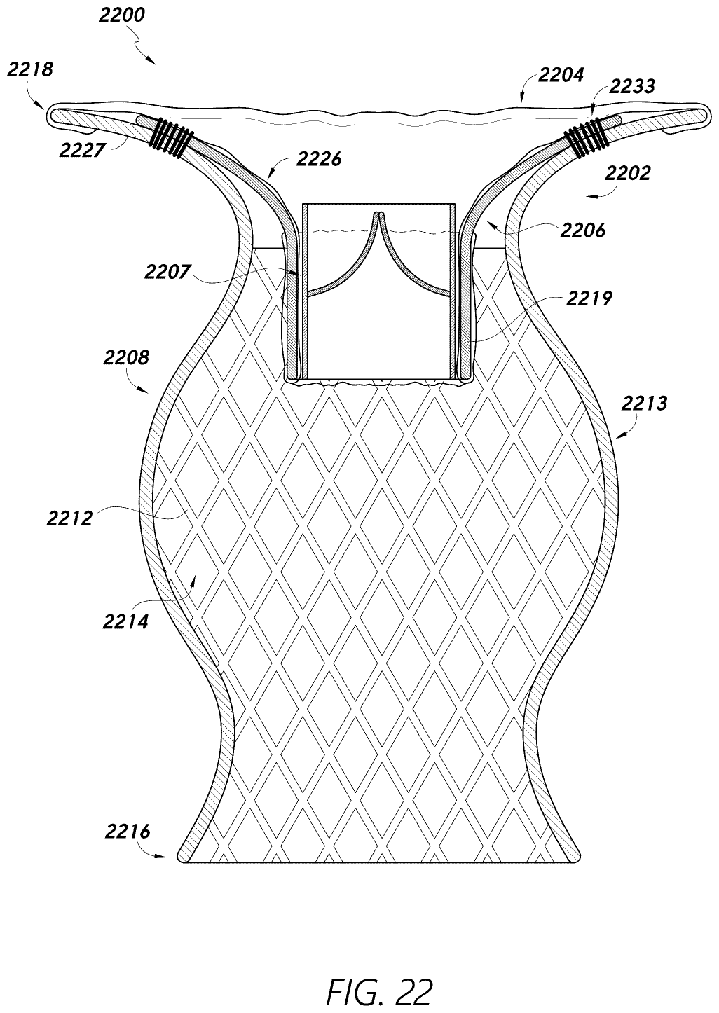

FIG. 22 provides a cross-sectional side view of an example implant 2200 configured to dock and/or support one or more prosthetic valves 2207 and/or valve components in accordance with one or more instances. The implant 2200 may be configured to dock the prosthetic valve 2207 at an inner frame 2206 of the implant 2200 and/or within a lumen of the inner frame 2206 of the implant 2200. The implant 2200 may comprise a frame 2202 and/or a sealing element 2204, which can include a skirt, covering, and/or similar device. The frame 2202 may be configured to form an inner frame 2206 and/or an outer frame 2208. The inner frame 2206 may form a first diameter that is less than a second diameter of the outer frame 2208. The inner frame 2206 and outer frame 2208 may be separate devices and/or may be joined and/or coupled by any suitable coupling means.

The outer frame 2208 may comprise one or more flared and/or elongate arms 2227 at a distal end 2218 of the implant 2000 and/or configured to extend outwardly and/or distally with respect to the outer frame 2208. In some examples, the outer frame 2208 may be configured to attach to and/or mate with a corresponding inner frame 2206. For example, the arms 2227 of the outer frame 2208 may be configured to attach to corresponding arms 2226 of the inner frame 2206. In some examples, the outer frame 2208 and/or inner frame 2206 may comprise various features configured to facilitate attachment and/or mating between the outer frame 2208 and the inner frame 2206. For example, the outer frame 2208 and/or inner frame 2206 may comprise one or more apertures and/or cavities configured to receive one or more binding and/or attachment tools 2233 and/or devices, which can include wires, strings, sutures, cords, and/or similar devices.

In some examples, the outer frame 2208 may have a generally cylindrical form and/or may at least partially enclose a complete circumference of the inner frame 2206 and/or of a portion of a sealing element 2204 attached to the outer frame 2208. The outer frame 2208 and/or inner frame 2206 may comprise a network of struts 2212, which can include wires, arms, bars, cords, walls, and/or similar components forming one or more cells 2214 and/or openings through the outer frame 2208 and/or inner frame 2206. The one or more cells 2214 may be configured to allow blood flow through the outer frame 2208. The cells 2214 may have any suitable shape and/or size. The outer frame 2208 and/or inner frame 2206 may form generally elongate cells 2214 extending approximately an entire length of the outer frame 2208 and/or from a proximal end 2216 to a distal end 2218 of the implant 2200. The one or more cells 2214 may be diamond shaped and/or may have any suitable shape. While the struts 2212 are shown having generally thick structures, the one or more struts 2212 may have wire-like and/or generally thin forms.

The implant 2200 may be configured for delivery and/or placement at an SVC and/or IVC of a heart. For example, the implant 2200 may be configured for placement at or near an in-flow junction of the SVC and/or IVC to the right atrium of the heart.

The implant 2200 may be configured to prevent blockage of one or more branching blood vessels (e.g., hepatic veins) when implanted within a main blood vessel (e.g., the IVC). For example, the implant 2200 may be configured to allow blood flow through the implant 2200 between the proximal end 2216 and the distal end 2218.

The implant 2200 may comprise one or more flared ends and/or arms 2227 at the distal end 2218 and/or proximal end 2216 of the implant 2200. In some examples, the implant 2200 may be laser-cut and/or may comprise one or more shape memory alloys (e.g., Nitinol).

The implant 2200 and/or outer frame 2208 may have any suitable shape. In some examples, the outer frame 2208 may have an hourglass shape and/or may comprise outward extending sides forming a bulge 2213 at or near a midpoint of the outer frame 2208. The proximal end 2216 and/or distal end 2218 of the implant 2200 may be generally flared and/or the outer frame 2208 may have generally inwardly extending sides between the bulge 2213 and the distal end 2218 and/or between the bulge 2213 and the proximal end 2216.

The inner frame 2206 may comprise one or more flared and/or elongate arms 2226 at a distal end 2218 of the inner frame 2206 and/or configured to extend outwardly and/or distally with respect to the inner frame 2206. In some examples, the inner frame 2206 may be configured to attach to and/or mate with a corresponding outer frame 2208

In some examples, the inner frame 2206 may have a generally cylindrical form and/or may be configured to fit at least partially within the outer frame 2208. The inner frame 2206 may comprise a generally cylindrical and/or tubular body 2219 portion comprising a network of struts 2212, which can include wires, arms, bars, cords, walls, and/or similar components forming one or more cells 2214 and/or openings through the inner frame 2206. The one or more cells 2214 may be configured to allow blood flow through the inner frame 2206. The cells 2214 may have any suitable shape and/or size. The inner frame 2206 may form generally elongate cells 2214 extending approximately an entire length of the outer frame 2208 and/or from a proximal end 2216 to a distal end 2218 of the implant 2200. The one or more cells 2214 may be diamond shaped and/or may have any suitable shape. While the struts 2212 are shown having generally thick structures, the one or more struts 2212 may have wire-like and/or generally thin forms.

The outer frame 2208, inner frame 2206, and/or one or more prosthetic valves may be delivered sequentially, simultaneously, and/or other otherwise. For example, a process may involve delivering the outer frame 2208 via one or more blood flow pathways to a target anatomical location (e.g., at a Thebesian valve and/or blood vessel ostium) and/or deploying and/or anchoring the outer frame 2208 at the target anatomical location. The outer frame 2208 and/or inner frame 2206 may be deployed at least partially within a blood vessel and/or chamber of a heart. For example, the outer frame 2208 may comprise various anchoring features and/or mechanisms and/or may comprise arms configured to facilitate deployment of the outer frame 2208. The inner frame 2206 may similarly be delivered via blood flow pathways to the deployed outer frame 2208 and/or may be mated and/or attached to the outer frame 2208. Mating and/or attaching the inner frame 2206 to the outer frame 2208 may involve extending and/or threading one or more attachment tools 2233 (e.g., cords) through apertures of the inner frame 2206 and/or outer frame 2208. The valve 2207 may then be docked and/or inserted into the inner frame 2206 and/or outer frame 2208. The valve 2207 may be deployed approximately at a center position of the inner frame 2206 and/or outer frame 2208.

### Additional Examples

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A cardiac implant device comprising a frame configured for placement at least partially within a blood vessel of a heart.

Example 2: The cardiac implant device of any example herein, in particular example 1, wherein the frame comprises an outer frame and an inner frame.

Example 3: The cardiac implant device of any example herein, in particular example 2, wherein the inner frame extends from the outer frame.

Example 4: The cardiac implant device of any example herein, in particular example 3, wherein the inner frame extends at an acute angle from the outer frame.

Example 5: The cardiac implant device of any example herein, in particular example 4, wherein at least a portion of the inner frame extends generally in parallel with the outer frame.

Example 6: The cardiac implant device of any example herein, in particular example 3, wherein the inner frame comprises one or more elongate arms.

Example 7: The cardiac implant device of any example herein, in particular example 6, wherein at least a first portion of the one or more elongate arms extends from at or near a distal end of the frame and extends towards a proximal end of the frame.

Example 8: The cardiac implant device of any example herein, in particular example 7, wherein at least a second portion of the one or more elongate arms extends from at or near the proximal end of the frame or a midsection of the frame and extends towards the distal end of the frame.

Example 9: The cardiac implant device of any example herein, in particular example 8, wherein each elongate arm of the first portion of the one or more elongate arms is disposed between two elongate arms of the second portion of the one or more elongate arms.

Example 10: The cardiac implant device of any example herein, in particular example 6, wherein at least a portion of the one or more elongate arms extends from at or near a proximal end of the frame or a midsection of the frame and extends towards a distal end of the frame.

Example 11: The cardiac implant device of any example herein, in particular example 3, wherein the frame comprises a U-shaped curve joining the outer frame and the inner frame.

Example 12: The cardiac implant device of any example herein, in particular example 11, wherein the U-shaped curve is disposed at a proximal end of the frame or at a midsection of the frame.

Example 13: The cardiac implant device of any example herein, in particular example 1, wherein the frame comprises a distal end configured to extend beyond the blood vessel and into a chamber of the heart.

Example 14: The cardiac implant device of any example herein, in particular example 13, wherein the distal end has a flared shape.

Example 15: The cardiac implant device of any example herein, in particular example 13, wherein the distal end comprises one or more triangular extensions.

Example 16: The cardiac implant device of any example herein, in particular example 13, wherein the distal end comprises one or more rectangular extensions.

Example 17: The cardiac implant device of any example herein, in particular example 13, wherein the frame further comprises a proximal end configured to extend below an inflow junction of the blood vessel and a branching blood vessel.

Example 18: The cardiac implant device of any example herein, in particular example 17, wherein the blood vessel is an inferior vena cava and the branching blood vessel is a hepatic vein.

Example 19: The cardiac implant device of any example herein, in particular example 17, wherein the proximal end has a flared form.

Example 20: The cardiac implant device of any example herein, in particular example 17, wherein the proximal end has a cylindrical form.

Example 21: The cardiac implant device of any example herein, in particular example 2, further comprising a sealing skirt configured to extend at least partially along the frame.

Example 22: The cardiac implant device of any example herein, in particular example 21, wherein the sealing skirt is configured to extend from a distal end of the outer frame to an inner surface of the inner frame.

Example 23: The cardiac implant device of any example herein, in particular example 22, wherein the sealing skirt comprises a distal portion and a proximal portion, and wherein the distal portion has a greater width than the proximal portion.

Example 24: The cardiac implant device of any example herein, in particular example 22, wherein the sealing skirt has a funnel shape.

Example 25: The cardiac implant device of any example herein, in particular example 22, wherein the sealing skirt has a mushroom shape.

Example 26: The cardiac implant device of any example herein, in particular example 21, wherein the sealing skirt has a disc shape.

Example 27: The cardiac implant device of any example herein, in particular example 21, wherein the sealing skirt is configured to contact a prosthetic valve.

Example 28: The cardiac implant device of any example herein, in particular example 1, wherein the frame comprises outward-extending sides forming a bulb.

Example 29: The cardiac implant device of any example herein, in particular example 1, wherein the frame comprises one or more outward-extending anchoring arms.

Example 30: The cardiac implant device of any example herein, in particular example 1, wherein the frame is configured to dock a prosthetic valve.

Example 31: The cardiac implant device of any example herein, in particular example 30, wherein the frame comprises one or more inward-extending arms configured to grasp the prosthetic valve.

Example 32: The cardiac implant device of any example herein, in particular example 1, further comprising one or more leaflets extends at least partially across a lumen of the frame.

Example 33: The cardiac implant device of any example herein, in particular example 1, further comprising one or more anchoring arms extending from a proximal end of the frame.

Example 34: The cardiac implant device of any example herein, in particular example 33, wherein a first anchoring arm of the one or more anchoring arms is configured to extend further into the blood vessel than a second anchoring arm of the one or more anchoring arms.

Example 35: The cardiac implant device of any example herein, in particular example 1, further comprising one or more anchoring arms extending from a distal end of the frame.

Example 36: The cardiac implant device of any example herein, in particular example 1, wherein the frame has an hourglass shape and comprises inward-extending sides at a midsection of the frame.

Example 37: The cardiac implant device of any example herein, in particular example 1, wherein the frame comprises a network of wire struts forming one or more cells.

Example 38: The cardiac implant device of any example herein, in particular example 37, wherein the network of wire struts forms relatively large cells at a midsection of the frame and forms relatively small cells at a distal end and proximal end of the frame.

Example 39: The cardiac implant device of any example herein, in particular example 1, wherein the frame has a tapered form and gradually decreases in diameter from a distal end of the frame to a proximal end of the frame.

Example 40: The cardiac implant device of any example herein, in particular example 1, further comprising one or more markers coupled to the frame.

Example 41: The cardiac implant device of any example herein, in particular example 2, wherein the inner frame and the outer frame are distinct components.

Example 42: The cardiac implant device of any example herein, in particular example 41, further comprising one or more cords configured to bind the inner frame to the outer frame.

Example 43: The cardiac implant device of any example herein, in particular example 42, wherein the inner frame or the outer frame comprises one or more apertures configured to receive the one or more cords.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

What is claimed is:

1. A cardiac implant device comprising:
a frame sized for placement at least partially within a blood vessel of a heart, the frame having a proximal end, a distal end, and a midsection, the frame comprising:
an outer frame; and
an inner frame, wherein at least a portion of the inner frame is joined to the outer frame at the distal end of the frame and wherein the inner frame extends away from the outer frame at the midsection of the frame to form a gap between the inner frame and the outer frame; and
a sealing skirt extending at least partially along the inner frame and the distal end of the frame.

2. The cardiac implant device of claim 1, wherein at least a portion of the inner frame extends in parallel with the outer frame.

3. The cardiac implant device of claim 1, wherein the inner frame and the outer frame are distinct components.

4. The cardiac implant device of claim 3, further comprising one or more cords configured to bind the inner frame to the outer frame.

5. The cardiac implant device of claim 1, wherein the sealing skirt does not extend along the outer frame at the midsection of the frame.

6. The cardiac implant device of claim 1, wherein the sealing skirt comprises a funnel-shaped end portion and a cylinder-shaped base portion.

7. The cardiac implant device of claim 6, wherein the end portion extends along the inner frame and the outer frame and the base portion extends along the inner frame and not along the outer frame.

8. The cardiac implant device of claim 1, wherein the frame further comprises a bulb-shaped base at the proximal end of the frame, wherein the bulb-shaped base has an increased width relative to the outer frame.

9. The cardiac implant device of claim 1, wherein the inner frame is not joined to the outer frame at the proximal end of the frame.

10. A cardiac implant device comprising:
a frame sized for placement at least partially within a blood vessel of a heart, the frame having a proximal end, a distal end, and a midsection, the frame comprising:
an outer frame having open cells to allow flow of blood through the outer frame; and
an inner frame, wherein at least a portion of the inner frame is joined to the outer frame at the distal end of the frame and wherein the inner frame extends away from the outer frame at the midsection of the frame to form a gap between the inner frame and the outer frame; and
a sealing skirt extending at least partially along the inner frame.

11. The cardiac implant device of claim 10, wherein the sealing skirt does not extend along the outer frame at the midsection of the frame.

12. The cardiac implant device of claim 10, wherein the sealing skirt comprises a funnel-shaped end portion and a cylinder-shaped base portion.

13. The cardiac implant device of claim 12, wherein the end portion extends along the inner frame and the outer frame and the base portion extends along the inner frame and not along the outer frame.

14. The cardiac implant device of claim 10, wherein the frame further comprises a bulb-shaped base at the proximal end of the frame, wherein the bulb-shaped base has an increased width relative to the outer frame.

15. The cardiac implant device of claim 10, wherein the inner frame is not joined to the outer frame at the proximal end of the frame.

16. A cardiac implant device comprising:
a frame sized for placement at least partially within a blood vessel of a heart, the frame having a proximal end, a distal end, and a midsection, the frame comprising:
an outer frame; and
an inner frame, wherein at least a portion of the inner frame is joined to the outer frame at the distal end of the frame and at the proximal end of the frame and wherein the inner frame extends away from the outer frame at the midsection of the frame to form a gap between the inner frame and the outer frame; and
a sealing skirt extending at least partially along the inner frame and the distal end of the frame.

17. The cardiac implant device of claim 16, wherein the sealing skirt does not extend along the outer frame at the midsection of the frame.

18. The cardiac implant device of claim 16, wherein the sealing skirt comprises a funnel-shaped end portion and a cylinder-shaped base portion.

19. The cardiac implant device of claim 18, wherein the end portion extends along the inner frame and the outer frame and the base portion extends along the inner frame and not along the outer frame.

20. The cardiac implant device of claim 16, wherein the inner frame is not joined to the outer frame at the proximal end of the frame.
